(19) United States
(12) Patent Application Publication
(10) Pub. No.: US 2012/0277161 A1
(43) Pub. Date: Nov. 1, 2012

(54) INHIBITION OF MULTIPLE CELL ACTIVATION PATHWAYS

(75) Inventors: Michael Valentine Agrez, Charlestown NSW (AU); Douglas Dorahy, Garden Suburb NSW (AU)

(73) Assignee: INTER-K PTY LIMITED, Newcastle NSW (AU)

(21) Appl. No.: 13/202,843

(22) PCT Filed: Feb. 23, 2010
(86) PCT No.: PCT/AU2010/000203

§ 371 Date: Oct. 25, 2011

(30) Foreign Application Priority Data
Feb. 23, 2009 (AU) 2009900762
Feb. 23, 2009 (AU) 2009900764
Jun. 19, 2009 (AU) 2009902829
Jun. 22, 2009 (JP) 2009902871
Aug. 20, 2009 (JP) 2009903938

(51) Int. Cl. A61K 38/02; A61P 35/00; C12N 9/99; C12N 5/09

(52) U.S. Cl. 514/19.3; 435/375; 435/184

(57) ABSTRACT

There is provided a method for inhibiting growth and/or proliferation of a cancer cell. The method comprises treating a cancer cell with an effective amount of a polypeptide providing a cytoplasmic binding domain of a β integrin subunit for binding of ERK2 to inhibit at least one protein kinase, other than a MAP kinase, in a cell activation pathway of the cancer cell. The protein kinases inhibited by the polypeptide may be selected from the group consisting of c-Raf, MEK 1 and kinases in the Src, PI3K, PKB/AKT and PKC families. Methods for the prophylaxis and treatment of cancer are also provided.

### FIELD OF THE INVENTION

The invention relates to inhibition of the growth and/or proliferation of cancer cells.

### BACKGROUND OF THE INVENTION

The spread of cancer cells involves tumour cell migration through the extracellular matrix scaffold, invasion of basement membranes, arrest of circulating tumour cells, and tumour cell extravasation and proliferation at metastatic sites. Detachment of cells from the primary tumour mass and modification of the peri-cellular environment aid penetration of tumour cells into blood and lymphatic vessels. It is the invasive and metastatic potential of tumour cells that ultimately dictates the fate of most patients suffering from malignant diseases. Hence, tumourigenesis can be viewed as a tissue remodelling process that reflects the ability of cancer cells to proliferate and digest surrounding matrix barriers. These events are thought to be regulated, at least in part, by cell adhesion molecules and matrix-degrading enzymes.

Cell adhesion receptors on the surface of cancer cells are involved in complex cell signalling which may regulate cell proliferation, migration, invasion and metastasis and several families of adhesion molecules that contribute to these events have now been identified including integrins, cadherins, the immunoglobulin superfamily, hyaluronate receptors, and mucins. In general, these cell surface molecules mediate both cell-cell and cell-matrix binding, the latter involving attachment of tumour cells to extracellular scaffolding molecules such as collagen, fibronectin and laminin.

Of all the families of cell adhesion molecules, the best-characterised is the family known as integrins. Integrins are involved in several fundamental processes including leucocyte recruitment, immune activation, thrombosis, wound healing, embryogenesis, virus internalisation and tumourigenesis. Integrins are transmembrane glycoproteins consisting of an alpha (α) and beta (β) chain in close association that provide a structural and functional bridge between extracellular matrix molecules and cytoskeletal components with the cell. The integrin family comprises 17 different α and 8 β subunits, and the αβ combinations are subsumed under 3 subfamilies.

Excluding the leucocyte integrin subfamily that is designated by the β2 nomenclature, the remaining integrins are arranged into two major subgroups, designated β1 and αv based on sharing common chains.

In the β1 subfamily, the β1 chain combines with any one of nine a chain members (α1-9), and the α chain which associates with β1 determines the matrix-binding specificity of that receptor. For example, α2β1 binds collagen and laminin, α3β1 binds collagen, laminin and fibronectin, and α5β1 binds fibronectin. In the αv subfamily of receptors, the abundant and promiscuous αv chain combines with any one of five β chains, and a distinguishing feature of αv integrins is that they all recognise and bind with high affinity to arginine-glycine-aspartate (RGD) (SEQ ID. No. 1) sequences present in the matrix molecules to which they adhere.

The current picture of integrins is that the N-terminal domains of α and β subunits combine to form a ligand-binding head. This head, containing the cation binding domains, is connected by two stalks representing both subunits, to the membrane-spanning segments and thus to the two cytoplasmic domains. The β subunits all show considerable similarity at the amino acid level. All have a molecular mass between 90 and 110 kDa, with the exception of β4 which is larger at 210 kDa. Similarly, they all contain 56 conserved cysteine residues, except for β4 which has 48. These cysteines are arranged in four repeating patterns which are thought to be linked internally by disulphide bonds. The α-subunits have a molecular mass ranging from 150-200 kDa. They exhibit a lower degree of similarity than the β chains, although all contain seven repeating amino acid sequences interspaced with non-repeating domains.

The β subunit cytoplasmic domain is required for linking integrins to the cytoskeleton. In many cases, this linkage is reflected in localisation to focal contacts, which is believed to lead to the assembly of signalling complexes that include α-actinin, talin, and focal adhesion kinase (FAK). At least three different regions that are required for focal contact localisation of β1 integrins have been delineated (Reszka et al, 1992). These regions contain conserved sequences that are also found in the cytoplasmic domains of the β2, β3, β5, β6 and β7 integrin subunits. The functional differences between these cytoplasmic domains with regard to their signalling capacity have not yet been established.

The integrin β6 subunit was first identified in cultured epithelial cells as part of the αvβ6 heterodimer, and the αvβ6 complex was shown to bind fibronectin in an arginine-glycine-aspartate (RGD)-dependent manner in human pancreatic carcinoma cells (Sheppard et al, 1990). The β6 subunit is composed of 788 amino acids and shares 34-51% sequence homology with other integrin subunits β1-β5. The β6 subunit also contains 9 potential glycosylation sites on the extracellular domain (Sheppard et al, 1990). The cytoplasmic domain differs from other subunits in that it is composed of a 41 amino acid region that is highly conserved among integrin subunits, and a unique 11 amino acid carboxy-terminal extension. The 11 amino acid extension has been shown not to be necessary for localisation of β6 to focal contacts. In fact, its removal appears to increase receptor localisation. However, removal of any of the three conserved regions identified as important for the localisation of β1 integrins to focal contacts (Reszka et al, 1992) has been shown to eliminate recruitment of β6 to focal contacts (Cone et al, 1994).

The integrin αvβ6 has previously been shown to enhance growth of colon cancer cells in vitro and in vivo (Agrez et al, 1994), and this growth-enhancing effect is due, at least in part, to αvβ6 mediated gelatinase B secretion (Agrez et al, 1999). What has made this epithelial-restricted integrin of particular interest in cancer is that it is either not expressed or expressed at very low levels on normal epithelial cells, but is highly expressed during wound healing and tumourigenesis, particularly at the invading edge of tumour cell islands (Breuss et al, 1995; Agrez et al, 1996).

Integrins can signal through the cell membrane in either direction. The extracellular binding activity of integrins can be regulated from the cell interior as, for example, by phosphorylation of integrin cytoplasmic domains (inside-out signalling), while the binding of the extracellular matrix (ECM) elicits signals that are transmitted into the cell (outside-in signalling). Outside-in signalling can be roughly divided into two descriptive categories. The first is ‘direct signalling’ in which ligation and clustering of integrins is the only extracellular stimulus. Thus, adhesion to ECM proteins can activate cytoplasmic tyrosine kinases (e.g., focal adhesion kinase FAK) and serine/threonine kinases (such as those in the mitogen-activated protein kinase (MAPK) cascade) and stimulate lipid metabolism (eg. phosphatidylinositol-4,5-biphosphate (P₁P₂) synthesis). The second category of integrin signalling is ‘collaborative signalling’, in which integrin-mediated cell adhesion modulates signalling events initiated through other types of receptors, particularly receptor tyrosine kinases that are activated by polypeptide growth factors. In all cases, however, integrin-mediated adhesion seems to be required for efficient transduction of signals into the cytosol or nucleus.

MAP kinases behave as a convergence point for diverse receptor-initiated signalling events at the plasma membrane. The core unit of MAP kinase pathways is a three-member protein kinase cascade in which MAP kinases are phosphorylated by MAP kinase kinases (MEKs) which are in turn phosphorylated by MAP kinase kinase kinases (e.g., Raf-1). Amongst the 12 member proteins of the MAP kinase family are the extracellular signal-regulated kinases (ERKs) (Boulton et al, 1991) activated by phosphorylation of tyrosine and threonine residues which is the type of activation common to all known MAP kinase isoforms. ERK 1/2 (44 kD and 42 kD MAPks, respectively) share 90% amino acid identity and are ubiquitous components of signal transduction pathways (Boulton et al, 1991). These serine/threonine kinases phosphorylate and modulate the function of many proteins with regulatory functions including other protein kinases (such as p90^{rsk}) cytoskeletal proteins (such as microtubule-associated phospholipase A₂), upstream regulators (such as the epidermal growth factor receptor and Ras exchange factor) and transcription factors (such as c-myc and Elk-1). ERKs play a major role in growth-promoting events, especially when the concentration of growth factors available to a cell is limited (Giancotti and Ruoslahti, 1999).

The two major growth signalling pathways activated through tyrosine kinase receptors at the cell membrane are the Ras-Raf-MEK-MAP kinase and the PI3 kinase/Akt/mTOR pathways. While PI3 kinases (PI3Ks) can be activated by interaction with the Ras proto-oncogene, it can be activated independently of Ras involvement, and PI3K activity alone is sufficient to promote cellular survival in the absence of trophic support and to block apoptosis induced by toxic stimuli. Hence, PI3K activity provides a parallel cell survival/activation pathway emanating from receptor tyrosine kinases. A diagram outlining kinase signaling (cell activation) pathways is shown in FIG. 1. As indicated in the diagram, signaling via MAP kinases and Akts can also occur through Src tyrosine kinase and Protein kinase C (PKC).

It is believed that of the compounds enrolled for Phase II and Phase III clinical trials only 11% manage to get through testing with some degree of efficacy, notwithstanding their side effects. This has led to an intense focus on inhibitors of PI3Ks to inhibit Akt mediated growth signalling. (Workman et al, 2007). PI3Ks and their lipid products promote survival downstream of extra cellular stimuli. Survival stimuli generally mediate intracellular signalling through ligation of transmembrane receptors which either possess intrinsic tyrosine kinase activity, are indirectly coupled to tyrosine kinases, or are coupled to seven transmembrane g-protein coupled receptors. Activation of these receptors results in the recruitment of PI3K isoforms to the inner surface of the plasma membrane as a result of ligand-regulated protein-protein interactions (Datta et al, 1999).

The family of PI3Ks is divided into several subgroups of which the Class I enzymes consists of the p85 adaptor subunit complexed with one of four p110 catalytic subunits (alpha, beta, delta, or gamma) and is capable of associating with receptor tyrosine kinases and oncoproteins (Zhao & Roberts, 2006). While PI3K gamma and delta are mainly expressed in haematopoietic tissues, alpha and beta are ubiquitously expressed.

The discovery in the late 1990s that firmly established the Class IA PI3Kinases (alpha, beta, or delta) as oncogenes was the finding that p110 alpha had been captured by a tumourigenic avian retrovirus rendering it oncogenic. Subsequently, an artificially activated form of p110 alpha was found to be capable of driving tumour formation when expressed in telomerase-immortalised human epithelial cells (reviewed in Zhao & Roberts, 2006). Hence, the p110 alpha isoform carries much of the signal from receptor tyrosine kinases and certain oncogenes such as Ras. Further, the PIK3CA gene, which encodes p110 alpha, is frequently mutated in a number of the most common forms of cancer, including colon, breast, prostate, liver and brain tumours.

While a number of targets downstream of PI3Ks have been implicated in suppression of apoptosis, c-Akt activation by PI3Ks is sufficient to block apoptosis induced by a number of death stimuli and Akt activity is required for growth factor-mediated survival. Akt was first implicated in signal transduction by the demonstration that the kinase activity of Akt is induced by growth factors such as basic fibroblast growth factor and PDGF. It is now known that a diverse array of physiological stimuli can induce Akt activity primarily in a PI3 Kinase-dependent manner. In turn, Akt regulates survival through the phosphorylation of multiple substrates involved in the regulation of apoptosis, for example, through phosphorylation of the Bcl-2 homolog Bad and caspase-9 (Datta et al, 1999).

Interestingly, colon cancers respond less well to the new anti-Akt compound, GK690693 in animal models than, for example, breast cancer which has a much higher frequency of PI3 Kinase/Akt mutations. Colon cancers have mutations of these kinase in at least 20% of tumours and a much higher incidence of mutation rates for Ras/Raf/BRaf. In fact, mutations of p110 alpha isoform of the PI3 Kinase subfamily is very common in cervical, breast and colon cancer (P. Workman, presentation at the HRMI Cancer Conference, Newcastle, NSW, September, 2008).

PI3K beta has also been shown to be required for de novo DNA synthesis in colon cancer cells (Benistant et al, 2000). Importantly, p110 alpha also functions in insulin signalling, whereas inhibition of p110 beta appears not to affect insulin signalling (Zhao & Roberts, 2006) making PI3K beta an attractive target.

Src kinases are cytoplasmic, membrane associated, non-receptor intracellular tyrosine kinases that mediate a variety of intracellular signalling pathways. They are cellular homologs of the products of the Rous sarcoma virus gene (v-Src), which is the mutated and activated version of a normal cellular gene (c-Src). There are nine members of this family of which Src, Fyn, and Yes are ubiquitously expressed, and Lck Hck, Fgr, Lyn and Blk have more tissue-restricted expression mainly in hematopoietic cells (Abram C L & Courtneidge S A, 2000). The remaining Src family member is Frk which is in its own subfamily. Src tyrosine kinases are known to be over expressed in a variety of tumour types, such as human colon adenocarcinoma (Windam T C et al, 2002; Haier J et al, 2002), breast cancer (Myoui A et al, 2003; Lu Y et al, 2003), pancreatic carcinoma (Lutz M P et al, 1998), and ovarian cancer (Budd R J et al, 1994; Weiner J R et al, 2003). Src family members are involved in numerous signalling pathways involved in proliferation, migration, tumour adhesion, and angiogenesis (Sato M et al, 2002) and mediate signalling from many types of receptors including receptor tyrosine kinases (RTKs), integrins, and G-protein-coupled receptors (Haier J et al, 2002). RTKs that signal through Src kinases include platelet-derived growth factor receptors (PDGFRs), epidermal growth factor receptors (EGFRs), and fibroblast growth factor receptors (Browaeys-Poly E et al, 2000). The Src family also appears to be required for growth factor-simulated DNA synthesis, particularly for growth factors with RTKs such as platelet-derived growth factor receptor and EGFR (Nanjundan M et al, 2003; Erpel T, 1996).

c-Src tyrosine kinase is the prototypical member of the Src family, and is involved in a variety of cell signalling events, regulating both cell proliferation and differentiation. Inhibition of c-Src is associated with decreased activation of cell growth and survival pathways. Src family kinases are required for the endomembrane activation of the Ras-MAPK pathway, where they phosphorylate and activate PLC-γ1. PLC-γ1 then activates RasGRP1, Ras guanine nucleotide exchange factor, thereby promoting Ras activation.

It has also been demonstrated that active c-Src kinase promotes survival of ovarian cancer cell lines and that inhibition of c-Src kinase sensitises ovarian cancer cells toward other chemotherapeutic agents (paclitaxel and cisplatin) (Pengetnze Y, 2003). For other cancer types, inhibition of c-Src kinase has been shown to result in significant anti-tumour activity against primary tumour growth and metastasis in an orthotopic nude mouse model for human pancreatic cancer.

Increased specific activity of c-Src is observed in >80% of colon adenocarcinomas relative to normal colonic mucosa (Bolen J B et al, 1987). Further increases in c-Src are seen in metastases relative to primary tumours. Thus, in the majority of colon tumour cells, c-Src is constitutively active. Recently, a subset of human colon tumours has been found to contain an activating mutation in the c-Src gene (Irby R B et al, 1999), although such mutations were not observed in other patient populations. Indeed, the increased specific activity of c-Src, whether due to infrequent mutation (Irby R B et al, 1999) or other mechanisms such as altered protein/protein association, is a hallmark of most colon tumours. It has also been reported that c-Src activity increases at progressive stages of the disease (Talamonti M S et al, 1993; Termulen P M et al, 1993) and is predictive of poor clinical prognosis (Allgayer H et al, 2002) suggesting that c-Src activation confers growth and/or survival advantages to metastatic colon tumour cells. Regardless of the mechanism of activation, there is substantial evidence suggesting that c-Src activation contributes to increased tumourigenicity of human colon cancer cell lines.

Using various colon tumour cell lines with different biologic properties and genetic alterations, it has further been shown that expression and activity of c-Src corresponds with resistance to anoikis. In particular, enforced expression of activated c-Src in subclones of SW480 cells (of low intrinsic c-Src expression and activity) increases resistance to anoikis, whereas decreased c-Src expression in HT29 colon cancer cells (of high c-Src expression and activity) by transfection with anti-sense c-Src expression vectors increases susceptibility to anoikis (Windham T C et al, 2002). Moreover, it has been postulated that c-Src activation may contribute to colon tumour progression and metastasis in part by activating Akt-mediated survival pathways that decrease sensitivity of detached cells to anoikis (Windham T C et al, 2002). In contrast, it has been reported that there is no alteration of ERK activity in response to increased or decreased c-Src activity in colon tumour cells (Windham T C et al, 2002).

In breast cancer, activation of the ERK/MAPK pathway has been shown to be a critical signal transduction event for estrogen-mediated proliferation. In contrast to the ability of herceptin (anti-HER2 monoclonal antibody) to inhibit estrogen-induced ERK activation, anti-epidermal growth factor receptor antibody has little effect (Venkateshwar G et al, 2002). However, inhibition of PKC delta-mediated signaling by the relatively specific PKC delta inhibitor, rottlerin, has been shown to block most of the estrogen-induced ERK activation (Venkateshwar et al, 2002) highlighting the importance of signaling “cross-talk” in cancer cells.

The PKC family consists of a number of serine-threonine kinases that are divided into three groups based on their activating factors. PKCs have been linked to carcinogenesis since PKC activators can act as tumor promoters and activation of the pKC alpha and beta isoenzymes (β1 and β2) have often been linked to the malignant phenotype. Indeed, PKC over-expression has been shown to stimulate Akt activity and suppress apoptosis induced by interleukin 3 withdrawal in myeloid cells (Weiqun L et al, 1999). Those investigators also demonstrated that PI3 Kinase inhibition suppressed PKC-mediated activation of Akt.

PKCs have, for instance, been reported to modulate the Inhibitor of Apoptosis Protein family (IAPs) that bind and potently inhibit the proteolytic activities of the pro-apoptotic caspases 3, 6 and 7 implicated in many different types of cancer including those with the highest mortality rates. PMA (phorbol myristate acetate) induced IAP expression appears to be a general feature of colon cancer cells and it has been shown (Wang Q et al) that PMA increases PKC delta activity, and blocking this enzyme prevents PMA from increasing IAP expression in colon cancer cells demonstrating a role for PKC-dependent signaling in prevention of apoptosis in human colon cancer cells.

The PKC beta isoforms (beta I and beta II) have also been reported to be an effective target for chemoprevention of colon cancer, and inhibition of PKC beta prevents invasion by rat intestinal epithelial cells mediated via activation of MEK signaling (Zhang J, 2004). Similarly, an inhibitor of PKC betaII has been shown to significantly reduce both tumor initiation in a colon cancer mouse model and tumor progression by inhibiting expression of pro-proliferative genes (Fields A P et al, 2009). PKC betaII has also been implicated in proliferation of the intestinal epithelium. For example, evidence has been provided for a direct role for PKC betaII in colonic epithelial cell proliferation and colon carcinogenesis, possibly through activation of the APC/beta catenin signaling pathway (Murray N R et al, 1999).

EGF-over-expressing invasive cancer cells have the ability to compensate for the loss of MAPK-mediated signaling through activation of PKC delta signaling for cell migration, which plays a major role in invasion and metastases (Kruger J S & Reddy K B, 2003). Those investigators have suggested that inhibition of MAPK and PKC delta signaling pathways should abrogate cell migration and invasion in EGFR-over-expressing human breast cancer cells.

The serine/threonine kinase Akt/PKB pathway functions as a cardinal nodal point for transducing extracellular (growth factor and insulin) and intracellular (receptor tyrosine kinases, Ras and Src) oncogenic signals. Moreover, ectopic expression of Akt, especially constitutively activated akt, is sufficient to induce oncogenic transformation of cells and tumor formation in transgenic mice as well as chemoresistance (Cheng J Q et al, 2005). Activated Akt is detectable and a poor prognostic factor for many types of cancer (reviewed in Targeting Akt in Cancer: Promise, Progress, and Potential Pitfalls: Dennis P A, AACR Education Book, 2008: 25-35). All the substrates of Akt have not yet been identified and the “critical substrates” can be cell type-specific. Hence, inhibition of individual downstream substrates of Akt may miss key substrates responsible for Akt-regulated survival or proliferation.

The finding that suppression of apoptosis by PKC alpha in myeloid progenitor cells correlates with its ability to activate endogenous Akt (Zhang et al, 1999) providing evidence of PKC-Akt “cross-talk”. It has been suggested that Akt3 (PKB gamma) may contribute to the more aggressive clinical phenotype characterized by estrogen receptor-negative breast cancers and androgen-insensitive prostate cancers (Nakatani K et al, 1999). Genetic inactivation of PTEN through either gene deletion or point mutation is reasonably common in metastatic prostate cancer and the resulting activation of PI3Ks and Akts provide a major therapeutic opportunity in cancer treatment (Majumber P K & Sellers W R, 2005). For example, in a prostate cancer cell line lacking the tumor suppressor PTEN, the basal enzymatic activity of PKB gamma has been found to be constitutively elevated and to represent the major active PKB isoform in these cells (Nakatani et al, 1999).

PKB beta (Akt2) is thought to be essential for cell survival and important in malignant transformation, and elevated PI3Kinase and Akt2 levels have been identified in 32 of 80 primary breast carcinomas (Sun M, 2001). This putative oncogene, Akt2, has also been found to be amplified and over-expressed in some human ovarian and pancreatic carcinomas (Cheng J Q et al, 1996).

The RAF serine/threonine family is composed of A-RAF, B-RAF and C-RAF (RAF-1). In contrast to the high incidence of B-raf mutations in human tumors, c-Raf mutations are rare due to its low basal activity. However, data showing the involvement of c-RAF in melanoma cell proliferation suggest that pan-specific RAF agents would be more efficacious against melanomas than B-Raf-specific drugs (Sebolt-Leopold J S, 2008).

The challenge faced in cancer therapy is how best to optimise the use of agents directed at specific kinases for tumors that harbor multiple genetic defects. MAPK pathway inhibitors (e.g., anti-MEK) are likely to find applicability across a wider range of tumors. However, RAS signals through multiple effectors, not just RAF. Consequently, activation by RAS of the PI3 Kinase/Akt survival signaling pathway may erode the therapeutic gain derived from shutting off MAPK activation in at least some tumours (Sebolt-Leopold J S, 2008). For example, it has been shown experimentally that the coexistence of an activating PI3K mutation reduces a K-RAS-mutated tumor's dependence on MEK/ERK signaling (reviewed by Sebolt-Leopold, 2008). Agents targeting upstream as well as downstream targets in the PI3K pathway, including PI3K and Akt, are logical candidates for combining with MEK and RAF inhibitors.

Recently, MAP kinases have been found to associate directly with the cytoplasmic domain of integrins, and binding domains of β3, β5 and β6 for binding of ERK1/2 have been characterised (see International Patent Application No. WO 2001/000677 and International Patent Application No. WO 2002/051993). The binding domain of β2 for binding of ERK1/2 was also reported in International Patent Application No. WO 2005/037308. Those patent applications show that inhibition of the β integrin-ERK1/2 binding interaction by a polypeptide providing the β integrin binding domain for the MAP kinase can inhibit growth of cancer cells. International Patent Application No. PCT/AU2004/001416 relates to the inhibition of growth of cancer cells in the absence of expression of the β integrin subunit.

There has been a major focus on combining different kinase inhibitors as a therapeutic approach to target different cell signaling/activation pathways as a treatment for cancer. However, this necessitates the identification of kinase mutations in individual cancers to enable a suitable combination of kinase inhibitors to be selected for treatment of the cancer. Single agents that target multiple cell signaling/activation pathways to inhibit “cross-talk” between the pathways would constitute a major advance in the treatment of cancer.

### SUMMARY OF THE INVENTION

The invention relates to the finding that an anti-cancer polypeptide providing a binding domain of a β integrin subunit for an extracellular signal-regulated kinase (ERK) of the mitogen activated protein (MAP) kinase family can inhibit the activity of protein kinase enzymes other than in the MAP kinase family, that are involved in a number of different cell activation pathways. This startling finding provides for the inhibition of multiple activation pathways in a cancer cell with a single therapeutic agent and thereby, the inhibition of cross-signalling or “cross-talk” between the pathways for the prophylaxis or treatment of cancer. More particularly, this finding provides for the inhibition of growth and proliferation of cancer cells that are mediated by aberrant or up-regulated activity of one or more cell activation pathways besides the Ras/Raf/MEK/MAPK pathway. The use of a polypeptide inhibitor of an ERK MAP kinase to inhibit the activity of a different class of kinase and particularly one involved in a cell activation pathway other than the Ras/Raf/MEK/MAPK pathway is entirely counter-intuitive, and represents a significant advance in the art.

Thus, broadly stated, the invention in one or more forms relates to a method for inhibiting a plurality of cell activation pathways in a cancer cell, comprising treating the cancer cell with an effective amount of a polypeptide providing a MAP kinase cytoplasmic binding domain of a 0 integrin subunit for binding of ERK2, or a variant or modified form of the binding domain, to which ERK2 binds.

In particular, in an aspect of the invention there is provided a method for inhibiting growth and/or proliferation of a cancer cell, comprising:

selecting an inhibitor for inhibiting at least one protein kinase in at least one cell activation pathway of the cancer cell other than a MAP kinase, the inhibitor being a polypeptide providing a MAP kinase cytoplasmic binding domain of a 0 integrin subunit for binding of ERK2, or a variant or modified form of the binding domain, to which ERK2 binds; and

treating the cancer cells with an effective amount of the polypeptide to inhibit the protein kinase.

The protein kinase(s) inhibited by the polypeptide can be selected from the group consisting of kinases in the Src, PI3K, Protein kinase B (PKB/AKT), and Protein kinase C (PKC) families. Surprisingly, it has further been found that c-Raf and MEK1 can also be inhibited by a polypeptide providing a MAP kinase cytoplasmic binding domain of a β integrin subunit for binding of ERK2.

Hence, in another aspect of the invention there is provided a method for inhibiting activity of at least one protein kinase, comprising:

selecting an inhibitor for inhibiting the protein kinase, the inhibitor being a polypeptide providing a MAP kinase cytoplasmic binding domain of a 0 integrin subunit for binding of ERK2, or a variant or modified form of the binding domain, to which ERK2 binds; and

contacting the target kinase with an effective amount of the polypeptide to inhibit the protein kinase, the protein kinase being selected from the group consisting of c-Raf, MEK1 and kinases in the Src, PI3K, Protein kinase B (PKB/AKT), and Protein kinase C (PKC) families.

In another aspect of the invention there is provided a method for inhibiting the activity of at least one protein kinase, comprising contacting the protein kinase with a polypeptide providing a MAP kinase cytoplasmic binding domain of a 0 integrin subunit for binding of ERK2, or a variant or modified form of the binding domain, to which ERK2 binds, the protein kinase being selected from the group consisting of c-RAF, MEK1, and kinases in the Src, PI3K, PKB and PKC families.

In another aspect of the invention there is provided a method for inhibiting a plurality of cell activation pathways in a cancer cell, comprising treating the cancer cell with an effective amount of at least one polypeptide providing a MAP kinase cytoplasmic binding domain of a β integrin subunit for binding of ERK2, or a variant or modified form of the binding domain, to which ERK2 binds.

In at least some embodiments, the cancer cell(s) can be treated with the polypeptide or a nucleic acid for expression of the polypeptide within the cells for effecting the treatment of the cells. Moreover, the polypeptide or nucleic acid can be presented by a dendrimer or coupled to another form of facilitator moiety for facilitating passage of the polypeptide or nucleic acid into the cytoplasm of the cancer cell, and all such embodiments are expressly encompassed by the invention.

As such, in yet another aspect of the invention there is provided a method for prophylaxis or treatment of cancer in a mammal, comprising administering to the mammal an effective amount of at least one dendrimer for inhibiting a plurality of cell activation pathways in cancer cells of the cancer, the dendrimer presenting at least one polypeptide providing a MAP kinase cytoplasmic binding domain of a 0 integrin subunit for binding of ERK2, or a variant or modified form of the binding domain, to which ERK2 binds.

Typically, the dendrimer and/or polypeptide is administered to inhibit the activity of at least two different protein kinases for inhibition of at least two activation pathways in the cancer cell(s).

When a dendrimer is administered the dendrimer will typically present more than 8 monomer units of the polypeptide.

Typically, the binding domain of the β integrin subunit incorporates an intervening amino acid linker sequence that links opposite end regions of the binding domain together wherein the linker sequence is not essential for the binding of ERK2. Moreover, one or more amino acids of the amino acid linker sequence may be deleted and/or differ in the polypeptide compared to the binding domain of the β integrin subunit.

Typically, all of the amino acids in the intervening amino acid sequence are deleted in the polypeptide compared to the binding domain.

The opposite end regions of the binding domain are defined by respective amino acid sequences, and typically, the amino acid sequence identity of the opposite end regions of the binding domain are unchanged in the polypeptide compared to the binding domain.

Typically, the β integrin subunit is expressed by the cancer cells of the cancer. However, in at least some embodiments, the cancer cells essentially do not express the β integrin subunit.

Typically, the cancer cells are treated with the dendrimer or polypeptide to inhibit at least one kinase in a cell activation pathway in the cancer cells other than, or besides, the Ras/Raf/MEK/MAPK pathway.

Most typically, the cells are treated with the dendrimer or polypeptide to inhibit the Ras/Raf/MEK/ERK activation pathway and at least one other cell activation pathway in the cells.

In at least some embodiments, the cells are treated with the dendrimer or polypeptide to inhibit one or more cell activation pathways selected from the group consisting of the PI3 kinase/Akt and PI3 kinase/Akt/mTOR pathways, and cell activation pathways involving one or more kinases in the Src, PKB/AKT and/or PKC kinase families.

The Src kinase(s) inhibited by the dendrimer or polypeptide can be one or more kinases selected from the group c-Src, c-Lyn, c-Yes and c-Fyn.

PKB is also known as the AKT protein kinase family, and the PKB kinase(s) inhibited by the polypeptide can be one or more kinases selected from the group consisting of PKB alpha (AKT1), PKB beta (AKT2), and PKB gamma (AKT3).

The PKC kinase(s) inhibited by the polypeptide can be one or more kinases selected from the group consisting of PKC alpha, PKC beta I, PKC beta II, PKC gamma and PKC delta.

The PI3K may be selected from the group of PI3 kinases consisting of the adaptor subunit (e.g., p85) complexed with a catalytic subunit (e.g., p110 alpha, beta, delta or gamma). In some embodiments, a mixture of these kinases may be inhibited by a method as described herein.

In at least some embodiments, the polypeptide will comprise, or consist of, an amino acid sequence selected from the group consisting of RSKAKWQTGTNPLYR (SEQ ID No: 2), RARAKWDTANNPLYK (SEQ ID No: 3), RSRARYEMASNPLYR (SEQ ID No: 4), KEKLKSQWNNDNPLFK (SEQ ID No: 5), RSKAKNPLYR (SEQ ID No: 6), RARAKNPLYK (SEQ ID No: 7), RSRARNPLYR (SEQ ID No: 8), and KEKLKNPLFK (SEQ ID No: 9).

The β integrin subunit will normally be selected from the group consisting of β2, β3, β5, and β6, and most usually, will be β6.

The binding domain of the β integrin subunit (or a variant or modified form of the binding domain) can be incorporated in a fusion protein, and the invention expressly extends to the use of such fusion proteins in a method embodied by the invention, whether presented in a dendrimer or not.

The dendrimer can be any type suitable for use in a method embodied by the invention. The dendrimer may, for example, have branched organic framework to which the binding domain (or modified or variant form thereof) is coupled, such as framework formed by poly(amidoamine) (PAMAM), tris(ethylene amine) ammonia or poly (propylene imine) (Astramol™). In other forms, the dendrimer can have framework incorporating polyamino acids forming branching units to which the peptide is coupled. In at least some embodiments, the dendrimer has a framework of branching units formed by polyamino acids.

Typically, the dendrimer will have a plurality of layers/generations of polyamino acid branching units to which the peptide is coupled. The polyamino acid branching units are normally formed by lysine residues. The respective units of the peptide presented by the dendrimer can provide the same or different binding domains (or variant forms thereof) of β integrin subunits to which ERK2 binds.

Typically, the dendrimer will present monomers of the peptide(s). The dendrimer can also have a core from which the branching framework of the dendrimer extends.

By the term “cancer” is meant any type of malignant, unregulated cell proliferation. The cancer can be selected from the group consisting of, but is not limited to, epithelial cell cancers, sarcomas, lymphomas and blood cell cancers, including leukemias such as myeloid leukemias, eosinophilic leukemias and granulocytic leukemias. For prophylaxis or treatment of a white blood cells cancer such as leukemia, the β subunit of the integrin may be β2 the expression of which is restricted to white blood cells (Hynes et al, 1992).

In addition, there is provided the use of a polypeptide providing a MAP kinase cytoplasmic binding domain of a β integrin subunit for binding of ERK2 to inhibit at least one target protein kinase in at least one cell activation pathway other than a MAP kinase, and thereby inhibit growth and/or proliferation of a cancer cell, or a variant or modified form of the binding domain, to which ERK2 binds, or a nucleic acid for expression of the polypeptide or the modified or variant form thereof in the cancer cell.

Further, there is provided the use of a polypeptide providing a MAP kinase cytoplasmic MAP kinase binding domain of a β integrin subunit for binding of ERK2 in the manufacture of a medicament for inhibiting at least one target protein kinase in at least one cell activation pathway other than a MAP kinase to inhibit growth and/or proliferation of a cancer cell, or a variant or modified form of the binding domain, to which ERK2 binds, or a nucleic acid for expression of the polypeptide or the modified or variant form thereof in the cancer cell.

The mammal can be any mammal treatable with a method of the invention. For instance, the mammal may be a member of the bovine, porcine, ovine or equine families, a laboratory test animal such as a mouse, rabbit, guinea pig, a cat or dog, or a primate or human being. Typically, the mammal will be a human being.

The features and advantages of invention will become further apparent from the following detailed description of non-limiting embodiments.

### BRIEF DESCRIPTION OF THE ACCOMPANYING DRAWINGS

FIG. 1 is a diagram illustrating cell activation pathways.

FIG. 2 is a schematic illustration of peptide dendrimer.

FIG. 3 (a) Shows a schematic illustration of a multiple antigen peptide dendrimer (MAP), incorporating eight peptide monomers. (b) An increase in the number of Lys branching units increases the number of surface amine groups.

FIG. 4 is a schematic illustration of a peptide dendrimer presenting 10 peptide monomers of the peptide RSKAKNPLYR (SEQ ID NO: 6) (referred to herein as dendrimer Dend 10-10(4)).

FIG. 5 is a graph showing dose response inhibition of c-Src tyrosine kinase activity by peptide RSKAKNPLYR (SEQ ID No: 4) in a cell-free assay.

FIG. 6 is a graph showing the efficacy of cisplatin and peptide AAVALLPAVLLALLARSKAKNPLYR (SEQ ID No: 10) (IK2) alone and in combination against chemotherapeutic drug-resistant ADDP human ovarian carcinoma cells compared to A2780 ovarian cancer cells treated with cisplatin alone.

FIGS. 7 (A) and (B) are graphs showing effect of oxaliplatin in combination with peptide AAVALLPAVLLALLARSKAKNPLYR (SEQ ID No: 10) (IK2) against ADDP human ovarian cancer cells.

FIG. 8 is a graph showing synergy between cisplatin and the peptide AAVALLPAVLLALLARSKAKNPLYR (SEQ ID No: 10) (IK2) against ADDP human ovarian cancer cells.

FIG. 9 is a graph showing synergy between cisplatin and the peptide AAVALLPAVLLALLARSKAKNPLYR (SEQ ID No: 10) (IK2) against HT29 human colon cancer cells.

FIG. 10 is a graph showing inhibition of ERK activity in HT29 colon cancer cells in a dose dependent manner by the peptide dendrimer Dend 8-10(4) presenting 8 monomer units of the peptide RSKAKNPLYR (SEQ ID No. 6).

FIG. 11 is a graph showing induction of apoptosis in human colon cancer cells by a peptide dendrimer presenting 10 monomer units of the peptide RSKAKNPLYR (SEQ ID No. 6) in which the peptide is comprised entirely of D amino acids and is pegylated (dendrimer Dend 10-10(4)DP).

FIG. 12 is a graph showing inhibition of proliferation of HT29 colon cancer cells by the dendrimer Dend 10-10(4) presenting 10 monomer units of the peptide RSKAKNPLYR (SEQ ID No. 6).

FIG. 13 is a graph showing the effect of dendrimers Dend 9-10(4) and Dend 12-10(4) (presenting 9 and 12 monomers of the peptide RSKAKNPLYR (SEQ ID No. 6), respectively) on proliferation of HT29 human colon cancer cells cultured for 48 hours.

FIG. 14 is a graph showing the efficacy of the dendrimer Dend 10-10(4)DP (identified as Mod. IK248) in inhibiting proliferation of HT29 colon cancer cells compared to cisplatin, irinotecan (CPT-11) and 5-fluorouracil (5FU).

FIG. 15 is a graph showing treatment of HT29 colon cancer cells with peptide dendrimer presenting 8 monomer units of the peptide RARAKNPLYK (SEQ ID No. 7) (Dend8-β3) (solid squares) or 8 monomers of peptide RSRARNPLYR (SEQ ID No. 8) (Dend8-β5) (solid diamonds).

FIG. 16 is a graph showing inhibition of HT29 colon cancer tumour growth in a BALB/c mouse model by the dendrimer Dend 10-10(4) (identified as IK248) (solid squares) compared to a vehicle only control (solid diamonds) when injected intra-tumorally.

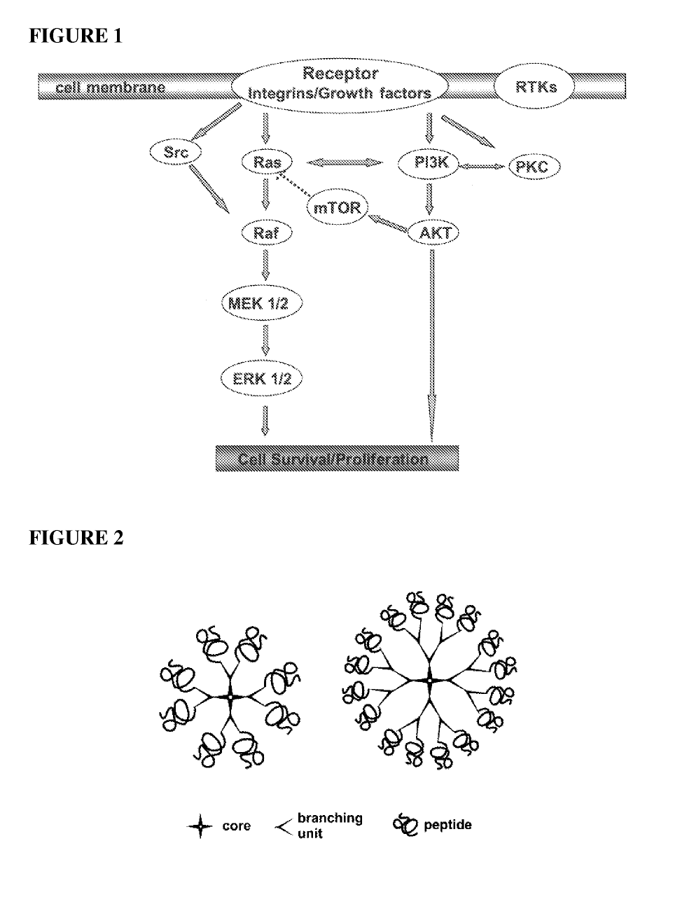

### DETAILED DESCRIPTION OF EXEMPLARY EMBODIMENTS OF THE INVENTION

Unexpectedly, it has been found that a peptide providing a MAP kinase cytoplasmic binding domain of a β integrin subunit for binding of ERK2 besides being an inhibitor of the ERK MAP kinase, is also an inhibitor of protein kinases in the Src and PI3K families as well as other protein kinases, including but not limited to c-RAF, MEK1, and kinases in the PKB (e.g., PKB alpha, PKB beta and PKB gamma) and protein kinase C (PKC) (e.g., PKC alpha, PKC beta I, PKC beta II, and PKC delta) families.

A polypeptide used in a method as described herein can provide the MAP kinase binding domain of the β-integrin subunit for binding of ERK2, or vary from the binding domain by one or more amino acids. The polypeptide may also, or alternatively, differ by one or more amino acids from one or both regions of the β-integrin subunit that flank the binding domain.

By the term “binding domain” is meant the minimum length of contiguous amino acid sequence of the β-integrin subunit required for binding of the MAP kinase substantially without compromising the optimum level of binding with the MAP kinase (e.g., ERK1/2). Moreover, the term “binding domain” includes those binding domains encoded by naturally occurring mutant and polymorphic alleles.

By the term “variant form” of the binding domain is meant an amino acid sequence that differs from the binding domain by one or more amino acids essentially without adversely effecting binding by the MAP kinase, and includes isolated or purified naturally occurring such sequences.

By the term “modified form” is meant an amino acid sequence in which the binding domain has been modified by one or more amino acid changes essentially without adversely affecting the binding by the MAP kinase.

By “MAP kinase” as used herein is meant a member of the mitogen activated protein kinase family (e.g., ERK1, ERK2, JNK and p38 isoforms) and excludes MAP kinase kinases and MAP kinase kinase kinase enzymes.

Variant and modified forms of the binding domain include derivatives and peptidomimetics of the binding domain. A variant or modified form of the binding domain will generally include 2 or more charged amino acid residues (each independently positively or negatively charged) and typically, a minimum of 3 positively charged amino acids (e.g., His, Lys, and/or Arg).

Typically, the polypeptide is a direct inhibitor of the protein kinase(s). That is, the polypeptide can inhibit the activity of the kinase(s) via the direct interaction of the polypeptide with the kinase(s).

Typically, the binding domain will have opposite end regions that are linked together by a number of contiguous intervening amino acids (i.e., an amino acid linker sequence) which are not essential for binding of ERK2 and can be deleted.

The provision of a polypeptide useful in a dendrimer or method embodied by the invention as described herein (e.g., a modified form of the binding domain of the (3 integrin subunit incorporating the binding domain) can be achieved by the addition, deletion and/or the substitution of one or more amino acids of the binding domain with another amino acid or amino acids. Inversion of amino acids and any other mutational change that results in alteration of an amino acid sequence are also encompassed. For example, one or more amino acids of the non-essential intervening amino acid linker sequence of the binding domain can be deleted or substituted for another amino acid or amino acids, (e.g., conservative amino acid substitution(s)). Such modified polypeptides can be prepared by introducing nucleotide changes in a nucleic acid sequence such that the desired amino acid changes are achieved upon expression of the mutagenised nucleic acid sequence, or for instance by synthesising an amino acid sequence incorporating the desired amino acid changes, which possibilities are well within the capability of the skilled addressee.

Further, a modified binding domain or polypeptide as described herein can incorporate an amino acid or amino acids not encoded by the genetic code, or amino acid analog(s). For example, D-amino acids rather than L-amino acids can be utilised. Indeed, a peptide useful in an embodiment of the invention may consist partly or entirely of D amino acids. D-peptides can be produced by chemical synthesis using techniques that are well-known in the art. Accordingly, in some embodiments, the peptide(s) may include L-amino acids, D-amino acids or a mixture of L- and D-amino acids. The synthesis of peptides including D-amino acids can inhibit peptidase activity (e.g., endopeptidase) and thereby enhance stability and increase the half-life of the peptide in vivo compared to the corresponding L-peptide.

Likewise, the N-terminal or C-terminal ends of the polypeptides/peptides can be modified to protect against or inhibit in vivo degradation (e.g., by peptidases). For instance, the C-terminus of the polypeptides can be amidated to protect against peptidase degradation. Alternatively, the N- or C-terminal end of a polypeptide as described herein can also be pegylated with a plurality of ethylene glycol monomer units to render it less resistant to degradation by proteases in vivo or to inhibit their clearance from the circulation via the kidneys. Methods for pegylation of polypeptides/peptides are well known in the art and all such methods are expressly encompassed. Typically, a pegylated polypeptide used in a method embodied by the invention will be coupled to 2 or more monomer units of polyethylene glycol (PEG) and generally, from about 2 to about 11 monomers of PEG (i.e., (PEG)n where n equals from 2 to 11). Most usually, n will be 2.

Substitution of an amino acid may involve a conservative or non-conservative amino acid substitution. By conservative amino acid substitution is meant replacing an amino acid residue with another amino acid having similar stereochemical properties (e.g., structure, charge, acidity or basicity characteristics) and which does not substantially adversely effect the binding activity of the binding domain. For example, a polar amino acid may be substituted with another polar amino acid, conservative amino acids changes being well known to the skilled addressee.

The sequence identity between amino acid sequences as described herein can be determined by comparing amino acids at each position in the sequences when the sequences are optimally aligned for the purpose of comparison. The sequences are considered the same at a position if the amino acids at that position are the same amino acid residue. Alignment of sequences can be performed using any suitable program or algorithm such as for instance, by the Needleman and Wunsch algorithm (Needleman and Wunsch, 1970). Computer assisted sequence alignment can be conveniently performed using standard software programs such as GAP which is part of the Wisconsin Package Version 10.1 (Genetics Computer Group, Madison, Wis., United States) using the default scoring matrix with a gap creation penalty of 50 and a gap extension penalty of 3. Other methods of alignment of sequences for comparison are also well known such as but not limited to the algorithms of Smith & Waterman, (1980) and Pearson & Lipman (1988), computerized implementation of such algorithms (e.g., BESTFIT, FASTA and BLAST), and by manual alignment and inspection.

Typically, a polypeptide useful in a dendrimer as described herein will have an overall amino acid sequence identity with the β integrin subunit of at least about 40% and more usually, at least about 50%, 60%, or 70% or greater and most preferably, at least about 80%, 90% or 95% sequence identity or greater. The sequence identity with the binding domain of the 0 integrin subunit may be greater than the overall amino acid sequence identity between the two sequences, and will usually be at least about 60%, 70% or 80% or greater, and more usually will be at least about 90%, or 95% or greater. However, it will be understood that the overall sequence identity of the polypeptide, or the sequence identity of the polypeptide with the binding domain, can be any specific value or range within the particular values specified above. For instance, the amino acid sequence identity of the polypeptide may be at least 66% or 75% or greater, and all such sequence identities and ranges are expressly encompassed by the invention.

A derivative of a polypeptide useful in a method embodied by the invention may be provided by cleavage cyclisation and/or coupling of the parent molecule with one or more additional moieties that improve solubility, lipophilic characteristics to enhance uptake by cells, stability or biological half-life, decreased cellular toxicity, or for instance to act as a label for subsequent detection or the like. A derivative may also result from post-translational or post-synthesis modification such as the attachment of carbohydrate moieties or chemical reaction(s) resulting in structural modification(s) such as the alkylation or acetylation of amino acid residues or other changes involving the formation of chemical bonds.

The term “polypeptide” is used interchangeably herein with peptide. For instance, it will be understood that peptide agents such as RSKAKNPLYR (SEQ ID No: 6) and KEKLKNPLFK (SEQ ID No: 9) fall within the scope of the term polypeptide.

Peptide dendrimers are particularly suitable for use in methods of the invention. Peptide dendrimers in at least some embodiments of the invention present units of the polypeptide inhibitor coupled to a branched framework of polyamino acids (typically lysine branching units). The dendrimer will typically have at least 3 layers/generations of amino acid branching units, the units of the polypeptide inhibitor being coupled to the outermost layer/generation of the amino acid branching units such that the dendrimer presents more than 8 units of the polypeptide. While monomer units of the polypeptide are preferred, in other embodiments, dendrimers incorporating multiple units of the polypeptide (multimers) (e.g., (RSKAKNPLYR)n ((SEQ ID No. 6)n), wherein n is the number of repeats of the polypeptide (typically 1-3)) coupled to polyamino acid branching units of the dendrimer may be utilized. Hence, the units of the polypeptide presented by the dendrimer can be monomer units, multimer units and/or mixtures of monomer and multimer units of the polypeptide.

The anti-cancer polypeptide can be bonded to the outermost layer/generation of polyamino acid branching units forming the framework of the dendrimer, or be synthetically assembled on the polyamino acid branching units of the dendrimer. More particularly, the synthesis of dendrimers useful in one or more methods embodied by the invention can be achieved by divergent or convergent synthesis strategies.

The divergent strategy is a direct approach by which the dendrimer is built stepwise in a continuous operation on a solid support through solid-phase synthesis. Stepwise synthesis involves synthesis of the branching core of the dendrimer followed by synthesis of the polypeptide inhibitor in a continuous manner. The divergent strategy is particularly suitable for the synthesis of dendrimers with a framework of a trifunctional acid (e.g., polyamino acid). Such solid phase synthesis schemes are the method of choice for the synthesis of lysine branching units where di-protected lysine is used to produce a branching framework of multiple levels of lysines. The diamino nature of lysine results in each additional level of lysine effectively doubling the number of sites upon which the polypeptide inhibitor may be synthesized directly.

The convergent strategy is an indirect, modular approach by which the polypeptide and branching core unit are prepared separately and then coupled together. Core units with branching framework used in the convergent synthesis of dendrimers are commercially available, and are typically formed from organic amino compounds such as poly(amidoamine) (PAMAM), tris(ethylene amine) ammonia or poly(propylene imine) (Astramol™) to which separately prepared inhibitor is normally covalently linked.

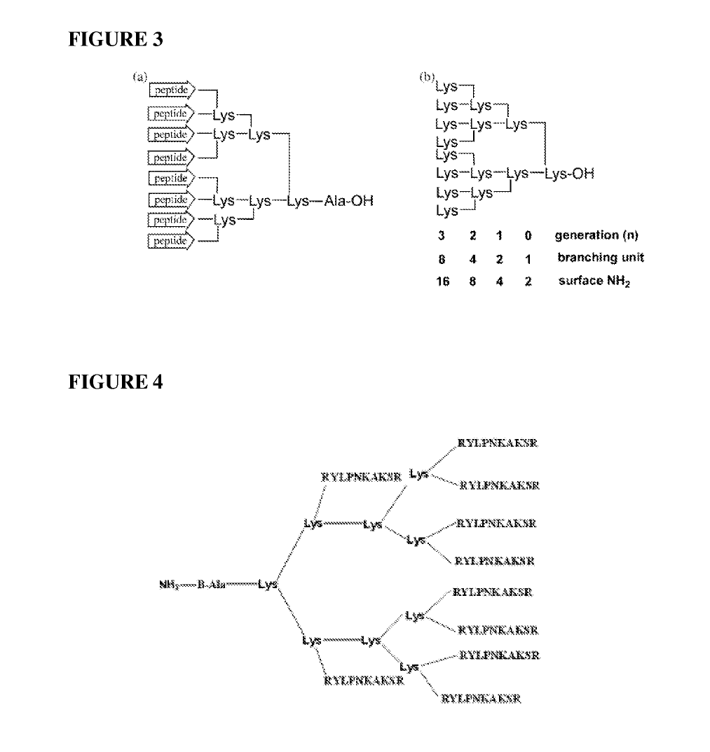

Suitable peptide dendrimer framework to which a polypeptide as described herein can be coupled, and methods for the provision of peptide dendrimers, are for example described in Lee et al, 2005; Sadler and Tam, 2002; and Cloninger, 2002, the entire contents of which are incorporated herein in their entirety by reference. Examples of peptide dendrimers of the type suitable for use in embodiments of the present invention are schematically illustrated in FIG. 2. and FIG. 3 (Sadler, K., and Tam, J. P., 2002), and in FIG. 4. Suitable dendrimers are also described in co-pending International Patent Application No. PCT/AU2009/000201, the contents of which is incorporated herein in its entirety by cross-reference.

A dendrimer used in a method embodied by the invention typically present more than 8 units of the polypeptide (e.g., 9, 10 or 12 units). While the polypeptide units will normally all be the same, mixtures of polypeptides as described herein can also be used. For example, half of the units of the polypeptide can provide the binding domain of the β6 integrin subunit for the ERK MAP kinase while the remaining units of the polypeptide present the binding domain of the β5 integrin subunit (or variant or modified forms of these binding domains). However, it will be understood that the ratio of the different anti-cancer polypeptide agents can be varied. The peptide(s) presented by a dendrimer used in a method embodied by the invention will also typically be N- or C-terminal protected against proteolytic degradation (e.g., by amidation, pegylation (i.e., the addition of PEG units) or the like). Methods such as pegylation of polypeptides are within the scope of the skilled addressee, and all such methods are expressly encompassed.

Typically, the polypeptide presented in the dendrimer in accordance with embodiments of the invention will have a length of about 60 amino acids or less. Usually, the polypeptide will have a length of more than 5 amino acids and will normally, be up to about 50 amino acids, 40, 35, 30, 25, 20 or 15 amino acids in length. In some forms, the polypeptide may have a length in a range of from 6, 7, 8, 9 or 10 amino acids up to about 14, 15, 16, 17, 18, 19, 20, or 25 amino acids. However, it will be understood that polypeptides of all specific lengths and length ranges within those identified above that are suitable for use in a dendrimer as described herein are expressly encompassed (e.g., 13 or 14 amino acids or from 10 to 15, 10 to 20 or 10 to 22 amino acids etc.).

The binding domain of the 0 integrin subunits β2, β3, β5 and β6 for the MAP kinase ERK2 are described in International Patent Application WO 2001/000677, and International Patent Application WO 2002/051993. The binding domain of the β2 integrin subunit for ERK2 is described in International Patent Application WO 2005/037308. The disclosures of all of these international patent applications are expressly incorporated herein by reference in their entirety. In particular, further polypeptide agents for inhibiting the binding of a β integrin subunit to a MAP kinase and which are suitable for being incorporated into a dendrimer as described herein are also described in those applications, as well as methodology for the localisation and characterization of the binding domains.

More particularly, the binding domain may be localised by assessing the capacity of respective overlapping peptide fragments of the cytoplasmic binding domain of a β integrin subunit for the ERK MAP kinase. The specific amino acid sequence which constitutes the binding domain may then be determined utilising progressively smaller peptide fragments. For this purpose, test peptides are readily synthesised to a desired length involving deletion of an amino acid or amino acids from one or both of the N-terminal and C-terminal ends of the larger peptide fragment(s), and tested for their ability to bind with the ERK MAP kinase. This process is repeated until the minimum length peptide capable of binding with the ERK MAP kinase substantially without compromising the optimum observed level of binding is identified.

The identification of amino acids that play an essential role in the ERK MAP kinase-β integrin interaction may be achieved with the use of further synthesised test peptides in which one or more amino acids of the sequence are deleted or substituted with a different amino acid or amino acids to determine the effect on the binding ability of the peptide. Typically, substitution mutagenesis will involve substitution of selected ones of the amino acid sequence with alanine or other neutrally charged amino acid.

Nucleotide and amino acid sequence data for the β6 integrin subunit for example is found in Sheppard et al, 1990. ERK1 and ERK2 have high overall amino acid sequence identity, with ERK1 having about 96% sequence identity to a 26 mer amino acid sequence of ERK2 providing the binding site for β6 (see International Patent Application No. WO 2002/051993. The nucleotide and amino acid sequence for ERK2 is for instance found in Boulton et al, 1991. Reference to such published data allows the ready design of polypeptides useful in the dendrimers described herein and the provision of the corresponding nucleic acid sequences encoding the polypeptides.

In order to constrain a polypeptide or other agent in a three dimensional conformation required for binding, it may be synthesised with side chain structures or incorporating cysteine residues which form a disulfide bridge. A polypeptide or other agent may also be cyclised to provide enhanced rigidity and thereby stability in vivo, and various such methods are known in the art.

As described above, a polypeptide useful in a method embodied by the invention can comprise, or consist of, the binding domain of the β integrin subunit, or a variant or modified form thereof in which one or more amino acids of the intervening amino acid sequence of the binding domain that are not essential for binding of the MAP kinase are deleted. As an example, the binding domain of β6 comprises the amino acid sequence RSKAKWQTGTNPLYR (SEQ ID No: 2). However, the intervening amino acid sequence WQTGT (SEQ ID No: 11) is not essential for binding of the MAP kinase ERK2. That is, even if the sequence WQTGT (SEQ ID No: 11) is deleted, a peptide with the amino acid sequence RSKAKNPLYR (SEQ ID No: 6) is still bound by ERK2. Similarly, the binding domains of β2, β3 and β5 for ERK2 are provided by KEKLKSQWNNDNPLFK (SEQ ID No. 5), RARAKWDTANNPLYK (SEQ ID No: 3) and RSRARYEMASNPLYR (SEQ ID No: 4), respectively. Deletion of the intervening sequences SQWNND (SEQ ID No. 12), WDTAN (SEQ ID No: 13) and YEMAS (SEQ ID No: 14) from these sequences yields the 10 mer peptides KEKLKNPLFK (SEQ ID No. 9), RARAKNPLYK (SEQ ID No: 7) and RSRARNPLYR (SEQ ID No: 8), all of which still bind to ERK2. As may be readily determined, the peptide RSKAKNPLYR (SEQ ID No. 6) has 80% sequence identity with peptide RSRARNPLYR (SEQ ID No: 8) and 70% sequence identity with the RARAKNPLYK (SEQ ID No. 7). Likewise, the peptide RSRARNPLYR (SEQ ID No. 8) has 70% sequence identity with peptide RARAKNPLYK (SEQ ID No. 7).

Alignment of the binding domains of β2, β3 and β5 and β6 results in the concensus scheme R/K x R/K x R/K-xxxxx NPL Y/F R/K wherein R/K is either arginine or lysine, Y/F is either tyrosine or phenylalanine, x may be any amino acid, and “-” (i.e., the dash) is an amino acid that is not essential and can be deleted, and a polypeptide utilized in a method embodied by the invention or a dendrimer as described herein may be represented by, or comprise, this consensus scheme. The amino acid designated by “-” can be a serine residue or may be another amino acid such as threonine, tyrosine, asparagine or glutamine. Typically, the polypeptide has an amino acid sequence represented by R/K x R/K*R/K-xx*x*NPL Y/F R/K wherein each * is independently a hydrophobic amino acid or an amino acid selected from the group consisting of serine, tyrosine and threonine. Hydrophobic amino acids are non-polar amino acids and examples include alanine, valine, leucine, isoleucine, and phenylalanine. The entire intervening amino acid sequence indicated by -xxxxx (or one or more of the amino acids of that sequence) may also be deleted such that the polypeptide comprises, or consists of, the sequence R/K x R/K x R/K NPL Y/F R/K.

Another way of achieving intracellular delivery of polypeptides, fusion proteins and the like as described herein is to use a “facilitator moiety” for facilitating passage or translocation of the polypeptide across the outer cell/plasma membrane into the cytoplasm of cells, such as a carrier peptide which has the capacity to deliver cargo molecules across cell membranes in an energy-independent manner. Carrier peptides that are known in the art include penetratin and variants or fragments thereof, human immunodeficiency virus Tat derived peptide, transportan derived peptide, and signal peptides.

Particularly suitable signal peptides are described in U.S. Pat. No. 5,807,746 the contents of which are incorporated herein in its entirety. Signal peptide for Kaposi fibroblast growth factor (K-FGF) consisting of, or incorporating, the amino acid sequence AAVALLPAVLLALLA (SEQ ID No: 15) or AAVALLPAVLLALLAP (SEQ ID No: 16) is preferred. It is not necessary that a signal peptide used in a method of the invention be a complete signal peptide, and fragments or modified or variant forms thereof and the like which retain the ability to pass across the outer cellular membrane to effect delivery of the attached peptide or other agent into the cytoplasm of the cell may be utilised.

Cationic peptides have also been used successfully to transfer macromolecules such as DNA into living cells and a 15 mer arginine peptide has been reported to be the preferred number of amino acid residues to mediate expression of DNA encoding green fluorescent protein and the β-galactosidase gene in cancer cell lines (Choi H S et al, 2006; Kim H H et al, 2003). The invention extends to the use of such cationic peptides as facilitator moieties for facilitating the passage into the target cancer cells of the polypeptide providing the binding domain of the β integrin subunit for the binding of ERK2 in accordance with the invention, or DNA encoding the polypeptide for expression of the polypeptide within the cells.

The invention in at least some embodiments also extends to coupling cationic peptide(s) such as a 15 mer arginine peptide (e.g., via a lysine bond) to a dendrimer presenting multiple units of the polypeptide (which may pegylated or unpegylated) providing the binding domain, or DNA encoding the polypeptide, for delivery/further assisting passage of the polypeptide or DNA into the target cancer cells, as a “double hit” strategy.

Rather than a carrier peptide, the facilitator moiety can be a lipid moiety or other non-peptide moiety which enhances cell membrane solubility of the selected anti-cancer peptide, such that passage of the peptide across the cell membrane is facilitated. The lipid moiety can for instance be selected from triglycerides, including mixed triglycerides. Fatty acids and particularly, C₁₆-C₂₀ fatty acids can also be used. Typically, the fatty acid will be a saturated fatty acid and most usually, stearic acid. The invention is not limited to the use of any such non-peptide facilitator molecule, and any molecule that provides the desired cell membrane solubility and which is physiologically acceptable can be used.

A polypeptide presenting the binding domain of a β integrin subunit for an ERK MAP kinase (or a variant or modified form of the binding domain) as described herein can be linked to the facilitator moiety in any conventionally known manner. For instance, the polypeptide can be linked directly to a carrier peptide through an amino acid linker sequence by a peptide bond or non-peptide covalent bond using a cross-linking reagent. Moreover, chemical ligation methods may be used to create a covalent bond between the carboxy terminal amino acid of the carrier peptide or linker sequence and a peptide comprising, or consisting of, the binding domain of the 0 integrin subunit for the ERK MAP kinase.

Targeting or delivery of polypeptides, nucleic acids or dendrimers to cancer cells as described herein may be achieved by coupling a targeting moiety such as a ligand (e.g., that binds to a receptor expressed by the cancer calls), or a binding peptide, an antibody or binding fragment thereof (such as Fab and F(ab)₂ fragments), to the facilitator moiety or directly to the dendrimer, polypeptide or the like. One approach employs coupling the facilitator moiety-peptide complex to integrin receptor-targeted peptides which target an extracellular integrin domain. For example, peptides with the sequence DLXXL (SEQ ID No: 17) can be used to target the extracellular domain of the β6 integrin subunit. Given that β6 expression enhances effective proteolysis at the cell surface by matrix metalloproteinase-9 (MMP-9) (Agrez M V et al, 1999), such targeting approaches include engineering an MMP-9 cleavage site between the targeting moiety and the carrier to facilitate internalisation of the carrier-agent complex. As another example, the ligand recognition motif for αVβ6 integrin, RTDLDSLRTYTL (SEQ ID No: 18) may be used in conjunction with or without an engineered MMP-9 cleavage site to deliver the facilitator moiety-peptide complex to the surface of the target cell.

Targeting of cancer cells in a method of the invention may also be achieved by coupling an antibody or binding peptide specific for the EGF receptor as are known in the art to the polypeptide or dendrimer (e.g., such as to the lysine (Lys) residue at the apex of the dendrimer illustrated in FIG. 4). Uptake into a cell can occur via a number of mechanisms, including via lysosomes which are rich in cathepsin, and targeting moieties employed can include a cathepsin cleavage site for release of the polypeptide or dendrimer to effect treatment of the cell. All such methods, dendrimers and polypeptides are expressly encompassed by the invention. Further, the polypeptide providing the binding domain of the β integrin subunit (and/or a variant or modified form of the binding domain) can also be pegylated as described above (whether the polypeptide is included in a dendrimer or not).

As another approach, liposomes, ghost bacterial cells, caveospheres, synthetic polymer agents, ultracentifuged nanoparticles and other anucleate nanoparticles (e.g., produced as a result of inactivating the genes that control normal bacterial cell division (De Boer P. A., 1989) may loaded with dendrimers or polypeptides as described herein and used for targeted delivery of the cargo to cancer cells (e.g., via labeling of the minicells, caveospheres or nanoparticles with bispecific antibodies, targeting peptides or the like as described above) (e.g., see also MacDiamid, JA, 2007). Such minicells and the like may be formulated for injection, or oral consumption for passage through the acid environment of the stomach for release and uptake of the dendrimer via the small intestine.

Still another approach is to load minicells as described above with nucleic acid encoding a polypeptide presenting the binding domain of the β integrin subunit for the ERK MAP kinase (or a variant or modified form of the binding domain) as described herein for delivery of the nucleic acid into cancer cells for expression of the polypeptide within the cells. As an alternative to minicells, caveospheres, bacteriophages, bacterial envelopes, recombinant vectors, and other conventional nanotechnology delivery methods can be used for delivery of the nucleic acid insert into the target cells. Any suitable vector incorporating the nucleic acid (eg., a genomic DNA or cDNA insert) for expression of the polypeptide in the cancer cells may be utilized, including plasmids. The expression vector may be designed for heterologous or homologous recombination events for integration of the nucleic acid into genomic DNA, and will typically include transcriptional regulatory control sequences to which the inserted nucleic acid sequence is operably linked. By “operably linked” is meant the nucleic acid insert is linked to the transcriptional regulatory control sequences for permitting transcription of the inserted sequence without a shift in the reading frame of the insert. Such transcriptional regulatory control sequences include promoters for facilitating binding of RNA polymerase to initiate transcription, expression control elements for enabling binding of ribosomes to transcribed mRNA, and enhancers for modulating promoter activity.

The use of fusion proteins incorporating a polypeptide which binds to the binding domain of an ERK MAP kinase for a β integrin subunit as described herein is also expressly provided for by the invention. Polypeptides and fusion proteins or the like can be chemically synthesised or produced using conventional recombinant techniques. Nucleic acid encoding a fusion protein may for instance be provided by joining separate DNA fragments encoding peptides or polypeptides having the desired amino acid sequence(s) by employing blunt-ended termini and oligonucleotide linkers, digestion to provide staggered termini as appropriate, and ligation of cohesive ends. Alternatively, PCR amplification of DNA fragments can be utilised employing primers which give rise to amplicons with complementary termini which can be subsequently ligated together (eg. see Ausubel et al. (1994) Current Protocols in Molecular Biology, USA, Vol. 1 and 2, John Wiley & Sons, 1992; Sambrook et al (1998) Molecular cloning: A Laboratory Manual, Second Ed., Cold Spring Harbour Laboratory Press, New York). Polypeptides and fusion proteins can be expressed in vitro and purified from cell culture for administration to a subject, or cells may be transfected with nucleic acid encoding a polypeptide or fusion protein for in vitro or in vivo expression thereof. The nucleic acid will typically first be introduced into a cloning vector and amplified in host cells, prior to the nucleic acid being excised and incorporated into a suitable expression vector for transfection of cells. Methods for the cloning, expression and purification of polypeptides useful in dendrimers as described herein are also well within the scope of the skilled addressee.

The toxicity profile of a polypeptide or dendrimer for use in a method embodied by the invention may be tested on cells by evaluation of cell morphology, trypan-blue exclusion, assessment of apoptosis and cell proliferation studies (e.g., cell counts, ³H-thymidine uptake and MTT assay).

Polypeptides as described herein (e.g., including in dendrimer form) can be co-administered with anti-sense therapy or one or more conventional anti-cancer compounds or drugs. By “co-administered” is meant simultaneous administration in the same formulation or in two different formulations by the same or different routes, or sequential administration by the same or different routes whereby the polypeptide(s) and drugs exert their effect over overlapping therapeutic windows.

Conventional chemotherapeutic drugs which may used in accordance with one or more embodiments of the invention can be selected from the group consisting of metal and non-metal based drugs. The metal complexes can be organic, inorganic, or mixed ligand co-ordination compounds or chelates. Transition metal complexes include for example complexes of platinum, palladium, copper, zinc, rhodium and ruthenium. Examples of platinum based chemotherapeutic drugs include cisplatin (cis-diamminedichloroplatinum (II)), oxaliplatin, ([Pt(1)xalto (1R), (2R)-diaminocyclohexane] complex), carboplatin (cis-diammine(1,1-cyclobutanedicarboxylato)platinum (II), and bleomycin. Further metal complexes are described for instance in U.S. Pat. No. 4,177,263 and International Patent Application No. WO 02/066435.

Examples of non-metal chemotherapeutic drugs include Paclitaxel, Gleevac, Docetaxel, Taxol, 5-fluorouracil, Doxorubicin, cyclophosphamide, Vincristine (Oncovin), Vinblastine, Vindesin, Camplothecin, Gemcitabine, Adriamycin, and topoisomerase inhibitors such as Irinotecan (CPT-11). Hence, a peptide as described herein can be co-administered with one or more of such conventional anti-cancer drugs or other drugs.

In particular, in the instance a drug resistant cancer is being treated, the dendrimer or polypeptide may be co-administered to the mammal in combination or in conjunction with the chemotherapeutic drug to which cells of the cancer are otherwise resistant. For example, inhibition of Src tyrosine kinase has been shown to enhance cytotoxicity of chemotherapeutic agents such as cisplatin in drug-sensitive ovarian cancer cells and to restore sensitivity in drug-resistant cells

In normal cells c-Src is maintained in an inactive configuration by multiple intramolecular interactions. The proto-oncogene c-Src is rarely mutated in human cancers although mutated c-Src that exhibits constitutive catalytic activity has been reported in small subsets of colon and endometrial cancers (reviewed by Ishizawar, R and Parsons, S. J, 2004). More commonly, this non-receptor tyrosine kinase exhibits elevated protein levels and increased activity of wild-type c-Src is seen in numerous types of human cancers. This arises from interactions between c-Src and many membrane bound receptors and cellular factors (e.g., growth factor receptors, integrins, steroid hormone receptors, G-protein receptors, focal adhesion kinase (FAK) and other adaptor proteins) (reviewed by Ishizawar and Parsons, 2004). As a consequence of these physical interactions, c-Src becomes transiently activated and phosphorylates downstream targets.

The Src family of cytoplasmic, membrane-associated non-receptor tyrosine kinases are upstream of MAP kinases and, therefore, ERK activation. Phosphorylation by c-Src of targets (for example) occurs in a unidirectional manner and is initiated by interactions between c-Src and the many membrane bound receptors and cellular factors near the plasma membrane as described above. As such c-Src and Src family members are critical mediators of multiple signaling pathways that regulate all stages of cancer progression (from initiation to metastasis) in multiple cell types.

The Src oncoprotein is extremely potent causing rapid transformation in cell culture and activated Src protein is over expressed in many human epithelial malignancies, particularly breast and colon cancers. Activated Src induces cellular invasion through a number of effectors, i.e., GTPase Rho and atypical protein kinase C. Moreover, one of the major alterations found in cells transformed by Src is that they can proliferate in the absence of external growth factors. The transcription factor Myc appears to be important in mediating Src's ability to cause cells to undergo unregulated cell proliferation.

Cell adhesion to extracellular matrix (ECM) proteins such as fibronectin and collagen is mediated by the binding of integrin receptors to ECM ligands. Integrin engagement leads to a number of intracellular signaling events, including the activation of Src family kinases (SFKs) and ERK1/2, responses that are dependent on the tyrosine phosphorylation and activation of focal adhesion kinase (FAK) (Miranti C K & Brugge J S, 2002). Further, aberrant integrin function and/or over-expression of focal adhesion kinase result in Src activation in focal adhesion complexes contributing to cell survival, and activating pathways that contribute to proliferation of some cell types (primarily through the Ras pathway) (Summy & Gallick, 2006). A functional interaction between integrins and c-Src has been recognised (Huveneers S et al, 2007).

A cancer treated in accordance with the invention will typically exhibit up-regulated activity of one or more of the kinase(s) inhibited by a dendrimer or polypeptide embodied by the invention. For example, the cancer can be a “Src” or a “PI3K” cancer. Moreover, in at least some embodiments, the cancer can be a drug resistant cancer (i.e., a cancer resistant to one or more other anti-cancer drugs., e.g., a multi-drug resistant cancer). In particular, the inhibition of c-Src and/or one or more other Src family kinases by a dendrimer or polypeptide in accordance with an embodiment of the invention may render a drug resistant cancer more susceptible, or otherwise sensitise the cancer to, conventional chemotherapeutic drug(s) to which the cancer is otherwise resistant.

By the term “Src cancer” is meant a cancer arising from, or associated with, aberrant and/or elevated levels of expression or activation of c-Src and/or one or more other Src family kinases. Similarly, the term “Src cancer cell” is meant a cancer cell arising from, or associated with, aberrant and/or elevated levels of expression or activation of c-Src and/or one or more other Src family kinases. Aberrant or elevated expression of activated the Src kinase may arise from the expression of mutant forms of c-Src (the mutation(s) causing constitutive activation of the protein) or for instance, as a result of interaction of c-Src with adjacent membrane bound receptor and/or cellular factors. In this instance, the membrane bound receptor or cellular factor may be constitutively activated (e.g., as a result of a mutation in the receptor or cellular factor).

Similarly, the term “PI3 kinase or PKC cancer” is meant a cancer arising from, or associated with, aberrant or elevated levels of expression and/or activation of a PI3 kinase or kinase in the PKC family. Likewise, by the terms “PI3 kinase cancer cell” and “PKC cancer cell” is meant a cancer cell arising from, or associated with, aberrant and/or elevated levels of expression and/or activation of a PI3 kinase or kinase in the PKC family. Aberrant or elevated expression and/or activation of a PI3 kinase or kinase in the PKC family may arise from the expression of mutant forms of the PI3K or kinase in the PKC family (the mutation(s) causing constitutive activation of the kinase) or for example, as a result of interaction of the PI3 kinase or PKC family member with cellular receptor(s) and/or factor(s).

The cancer treated by a method of the invention may for instance be selected from the group consisting of carcinomas, sarcomas, lymphomas, solid tumors, head and neck cancers, blood cell cancers, leukaemias, myeloid leukaemias, eosinophilic leukaemias, granulocytic leukaemias, and cancer of the liver, tongue, salivary glands, gums, floor and other areas of the mouth, oropharynx, nasopharynx, hypopharynx and other oral cavities, oesophagus, gastrointestinal tract, stomach, small intestine, duodenum, colon, colonrectum, rectum, gallbladder, pancreas, larynx, trachea, bronchus, lung (including non-small cell lung carcinoma), breast, uterus, cervix, ovary, vagina, vulva, prostate, testes, penis, bladder, kidney, thyroid, bone marrow, and skin (including melanoma). Typically, the cancer will be an epithelium cancer and most usually, a non-dermal cancer. Most usually, the cancer will be selected from the group consisting of lung cancers, colon cancers, pancreatic cancers, breast cancers, colon adenocarcinomas and ovarian cancers.

The polypeptide used will typically be formulated into a pharmaceutical composition comprising a pharmaceutically acceptable carrier and/or excipient for administration to the intended subject. The peptide can be administered orally, intravenously, parenterally, rectally, subcutaneously, by infusion, topically such as in the treatment of skin cancers, intramuscularly, intraperitonealy, intranasally and by any other route deemed appropriate. The pharmaceutical composition can for example be in the form of a liquid, suspension, emulsion, syrup, cream, ingestable tablet, capsule, pill, suppository, powder, troche, elixir, or other form that is appropriate for the selected route of administration.

In particular, pharmaceutical compositions embodied by the invention include aqueous solutions. Injectable compositions will be fluid to the extent that syringability exists and typically, will normally stable for a predetermined period to provide for storage after manufacture. Moreover, pharmaceutically acceptable carriers include any suitable conventionally known solvents, dispersion media, physiological saline and isotonic preparations or solutions, and surfactants. Suitable dispersion media can for example contain one or more of ethanol, polyols (e.g., glycerol, propylene glycol, liquid polyethylene glycol and the like), vegetable oils and mixtures thereof.

For oral administration, any orally acceptable carrier can be used. In particular, the polypeptide can be formulated with an inert diluent, an assimilable edible carrier or it may be enclosed in a hard or soft shell gelatin capsule.

Topically acceptable carriers conventionally used for forming creams, lotions or ointments for internal or external application can be employed. Such compositions can be applied directly to a site to be treated or via by dressings and the like impregnated with the composition.

A pharmaceutical composition as described herein can also incorporate one or more preservatives suitable for in vivo and/or topical administration such as parabens, chlorobutanol, phenol, sorbic acid, and thimerosal. In addition, prolonged absorption of the composition may be brought about by the use in the compositions of agents for delaying absorption such as aluminium monosterate and gelatin. Tablets, troches, pills, capsules and the like containing the polypeptide can also contain one or more of the following: a binder such as gum tragacanth, acacia, corn starch or gelatin; a disintegrating agent such as corn starch, potato starch or alginic acid; a lubricant such as magnesium sterate; a sweetening agent such as sucrose, lactose or saccharin; and a flavouring agent.

The use of ingredients and media as described above in pharmaceutical compositions is well known. Except insofar as any conventional media or ingredient is incompatible with the dendrimer, use thereof in therapeutic and prophylactic compositions as described herein is included.

It is particularly preferred to formulate parenteral compositions in dosage unit form for ease of administration and uniformity of dosage. Dosage unit form as used herein is to be taken to mean physically discrete units suited as unitary dosages for the subject to be treated, each unit containing a predetermined quantity of active agent calculated to produce the desired therapeutic or prophylactic effect in association with the relevant carrier used. When the dosage unit form is for example, a capsule, tablet or pill, various ingredients may be used as coatings (e.g., shellac, sugars or both) to otherwise modify the physical form of the dosage unit or to facilitate administration to the individual.

A pharmaceutical composition will generally contain at least about 1% by weight of the polypeptide. The percentage may of course be varied and can conveniently be between about 5% to about 80% w/w of the composition or preparation. As will be understood, the amount of the peptide in the composition will be such that a suitable effective dosage will be delivered to the subject taking into account the proposed route of administration. Preferred oral compositions embodied by the invention will contain between about 0.1 μg and 15 g of the polypeptide.

The dosage of the polypeptide will depend on a number of factors including whether the polypeptide is to be administered for prophylactic or therapeutic use, the condition for which the polypeptide is intended to be administered, the severity of the condition, the age of the subject, and related factors including weight and general health of the individual as may be determined by the physician or attendant in accordance with accepted principles. For instance, a low dosage may initially be given which is subsequently increased at each administration following evaluation of the individual's response. Similarly, the frequency of administration may be determined in the same way that is, by continuously monitoring the individual's response between each dosage and if necessary, increasing the frequency of administration or alternatively, reducing the frequency of administration.

Typically, the polypeptide will be administered in accordance with a method of the invention to provide a dosage of the polypeptide of up to about 100 mg/kg body weight of the individual, more usually in a range up to about 50 mg/kg body weight, and most usually in a range of about 5 mg/kg to 40 mg/kg body weight. In at least some embodiments, the polypeptide will be administered to provide a dosage of the polypeptide in a range of from about 5 to 25 mg/kg body weight, usually in a range of from about 5 mg/kg to about 20 mg/kg and more usually, in a range of from 10 mg/kg to about 20 mg/kg. When administered orally in dendrimer form, up to about 20 g of the dendrimer may be administered per day, (e.g., 4 oral doses per day, each dose comprising 5 g of the dendrimer).

With respect to intravenous routes, particularly suitable routes are via injection into blood vessels which supply a tumour or a cancer in to be treated in particular organs. In particular, the polypeptide, dendrimer, fusion protein or the like can be delivered into isolated organs, limbs and tissue by any suitable infusion or perfusion techniques. The polypeptide may also be delivered into cavities such for example the pleural or peritoneal cavity, or be injected directly into tumour tissue. Suitable pharmaceutically acceptable carriers and formulations useful in compositions of the present invention can for instance, be found in handbooks and texts well known to the skilled addressee, such as “Remington: The Science and Practice of Pharmacy (Mack Publishing Co., 1995)”, the contents of which is incorporated herein in its entirety by reference.

The present invention will be described herein after with reference to a number of non-limiting Examples.

### Example 1

### Inhibition of c-Src by RSKAKNPLYR (SEQ ID No. 4)

The RSKAKNPLYR peptide (50 μM) (SEQ ID No: 4) (designated 10(4)) was assayed for inhibitory activity against the MAP kinase ERK2, cellular Src tyrosine kinase (c-Src) and the tyrosine kinases Lyn and Yes (at equivalent activity concentrations). The assay conditions for each kinase were as follows (Upstate Kinase Profiling Services, Dundee, Scotland).

### 1. Kinase Activity Assays

1.1 c-Src

In a final reaction volume of 25 μL, c-SRC (h) (5-10 mU) is incubated with 8 mM MOPS pH 7.0, 0.2 mM EDTA, 250 μM KVEKIGEGTYGVVYK (SEQ ID No. 19) (Cdc2 peptide), 10 mM MgAcetate and [γ-33P-ATP] (specific activity approx. 500 cpm/pmol, concentration as required). The reaction is initiated by the addition of the MgATP mix. After incubation for 40 minutes at room temperature, the reaction is stopped by the addition of 5 μL of a 3% phosphoric acid solution. 10 μL of the reaction is then spotted onto a P30 filtermat and washed three times for 5 minutes in 75 mM phosphoric acid and once in methanol prior to drying and scintillation counting.

### 1.2 ERK2

In a final reaction volume of 25 μL, human ERK2 (5-10 mU) is incubated with 25 mM Tris pH 7.5, 0.02 mM EGTA, 0.33 mg/mL myelin basic protein, 10 mM MgAcetate and [γ-33P-ATP] (specific activity approx. 500 cpm/pmol, concentration as required). The reaction is initiated by the addition of the MgATP mix. After incubation for 40 minutes at room temperature, the reaction is stopped by the addition of 5 μL of a 3% phosphoric acid solution. 10 μL of the reaction is then spotted onto a P30 filtermat and washed three times for 5 minutes in 75 mM phosphoric acid and once in methanol prior to drying and scintillation counting.

### 1.3 Lyn

In a final reaction volume of 25 μl, human Lyn is incubated with the RSKAKNPLYR peptide (SEQ ID No: 6) in 50 mM Tris buffer pH 7.5, 0.1 mM EGTA, 0.1 mM Na3VO4, 1% mercaptoethanol, 0.1 mg/ml poly(Glu,Tyr) 4:1, 10 mM Mg acetate and [γ-33P-ATP] (specific activity approx. 500 cpm/pmol). The reaction was initiated by the addition of the MgATP mix, incubated for 40 minutes at room temperature prior to being stopped and kinase activity assessed by scintillation counting as per the protocol described for c-Src described in Example 1.1.

### 1.4 Yes

In a final reaction volume of 25 μl, human Yes is incubated with the RSKAKNPLYR peptide (SEQ ID No: 6) in 8 mM MOPS buffer pH 7.0, 0.2 mM EDTA, 0.1 mg/ml poly(Glu,Tyr) 4:1, 10 mM Mg acetate and [γ-33P-ATP] (specific activity approx. 500 cpm/pmol). The reaction is initiated by the addition of the MgATP mix, incubated for 40 minutes at room temperature prior to being stopped and kinase activity assessed by scintillation counting again as per the protocol described for c-Src in Example 1.1.

### 1.5 Results

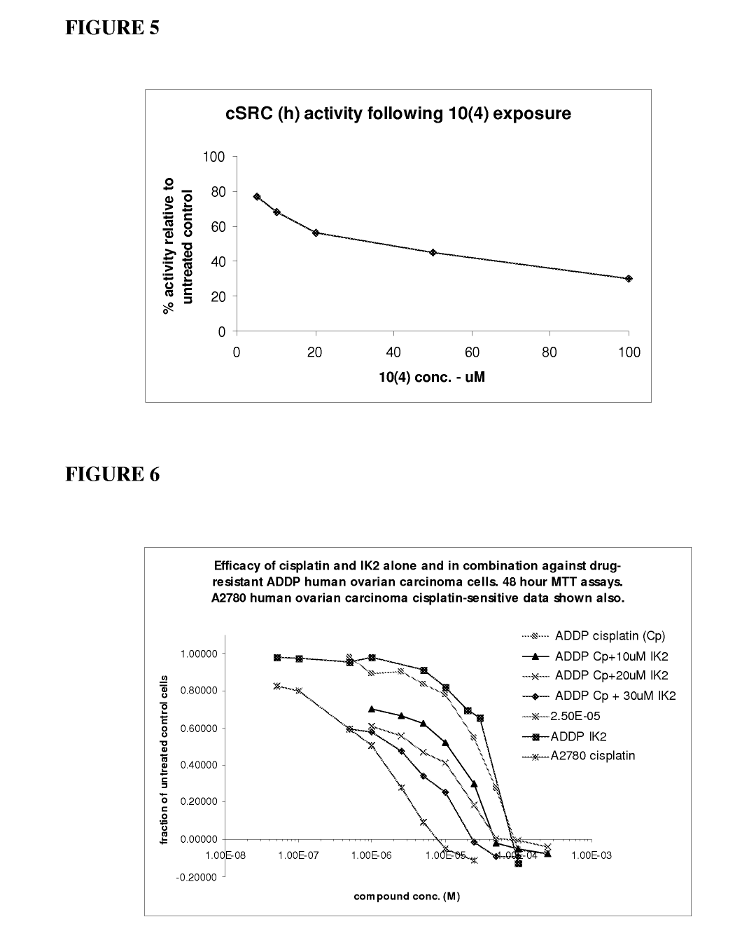

The results were expressed as percentage activity relative to control (activated c-Src and substrate alone in the absence of peptide inhibitor). The RSKAKNPLYR peptide (SEQ ID No: 6) (10(4) peptide) significantly inhibited the activity of c-Src (by approx. 57% activity relative to control). Relatively low level inhibition of Lyn and Yes activity were observed. Dose dependent inhibition of c-Src by the peptide RSKAKNPLYR (SEQ ID No: 4) is shown in FIG. 5.

### Example 2

### Inhibition of PI3 Kinases by RSKAKNPLYR (SEQ ID No. 6)

A peptide dendrimer of the type shown in FIG. 4 and presenting 10 monomer units of the peptide RSKAKNPLYR (SEQ ID No. 6) was assayed for inhibitory activity against ERK2 and the PI3Ks PI3K beta and PI3K gamma. The dendrimer is referred to herein as dendrimer IK248B (or Dend 10 10(4)). The treatment protocols were as described below (Upstate Kinase Profiling Services, Dundee, Scotland).

### 2. Kinase Activity Assays

### 2.1 ERK2

The activity of ERK2 was assayed as described in Example 1.2.

### 2.2 PI3K

In a final reaction volume of 20 μL, the test PI3K is incubated in assay buffer containing 10 μM phosphatidylinositol-4,5-bisphosphate and MgATP. The reaction is initiated by the addition of the MgATP mix. After incubation for 30 minutes at room temperature, the reaction is stopped by the addition of 5 μL of stop solution containing EDTA and biotinylated phosphatidylinositol-3,4,5-trisphosphate. Finally, 5 μL of detection buffer is added (containing europium-labelled anti-GST monoclonal antibody, GST-tagged GRP1 PH domain and streptavidin-allophycocyanin). The test plate is then read in time-resolved fluorescence mode and the homogenous time-resolved fluorescence (HTRF) signal is determined according to the formula HTRF=10000×(Em665 nm/Em620 nm).

### 2.3 Results

Results were expressed as a percentage activity of control (PI3K or ERK2 in the absence of dendrimer). At a final concentration of 50 μM, dendrimer Dend 10-10(4) inhibited ERK2 to 47% activity relative to control cells whereas PIK3 beta and PIK3 gamma were inhibited to 83% and 11% activity relative to control by the dendrimer.

In another study in which the RSKAKNPLYR peptides (SEQ ID No. 6) of the dendrimer were bipegylated at their C-terminal ends with two ethylene glycol units and were comprised entirely of D-amino acids (identified herein as dendrimer Dend 10 D-10(4)DP), the activity of PI3K beta was nearly completely abrogated by the dendrimer (approx. 92% inhibition; 20 μM final concentration).

### Example 3

### Inhibition of Further Kinases

The ability of the peptide dendrimer Dend 10-10(4)DP described in Example 2.3 to inhibit the activity of further kinase enzymes was evaluated. The ability of a further dendrimer of the type shown in FIG. 4 presenting 10 monomer units of the β5 integrin derived peptide RSRARNPLYR (SEQ ID No. 8) (Dend 10β5) to inhibit ERK2 and MEK1 was also evaluated. The treatment protocols employed are set out below (Upstate Kinase Profiling Services, Dundee, Scotland).

3.1 e-RAF

In a final reaction volume of 25 μL, c-RAF (h) (5-10 mU) is incubated with 25 mM Tris pH 7.5, 0.02 mM EGTA, 0.66 mg/mL myelin basic protein, 10 mM MgAcetate and [γ-₃₃P-ATP] (specific activity approx. 500 cpm/pmol, concentration as required). The reaction is initiated by the addition of the MgATP mix. After incubation for 40 minutes at room temperature, the reaction is stopped by the addition of 5 μL of a 3% phosphoric acid solution. 10 μL of the reaction is then spotted onto a P30 filtermat and washed three times for 5 minutes in 75 mM phosphoric acid and once in methanol prior to drying and scintillation counting.

### 3.2 ERK2 (h)

The activity of ERK2 was assayed as described in Example 1.2.

### 3.3 MEK1 (h)

In a final reaction volume of 25 μL, MEK1 (h) (1-5 mU) is incubated with 50 mM Tris pH 7.5, 0.2 mM EGTA, 0.1% mercaptoethanol, 0.01% Brij-35, 1 μM inactive ERK2 (m), 10 mM MgAcetate and cold ATP (concentration as required). The reaction is initiated by the addition of the MgATP. After incubation for 40 minutes at room temperature, 5 μL of this incubation mix is used to initiate an ERK2 (m) assay.

### 3.4 PKB Kinases (h)

In a final reaction volume of 25 μL, PKB(h) (5-10 mU) is incubated with 8 mM MOPS pH 7.0, 0.2 mM EDTA, 30 μM GRPRTSSFAEGKK (SEQ ID No. 26), 10 mM MgAcetate and [γ-₃₃P-ATP] (specific activity approx. 500 cpm/pmol, concentration as required). The reaction is initiated by the addition of the MgATP mix. After incubation for 40 minutes at room temperature, the reaction is stopped by the addition of 5 μL of a 3% phosphoric acid solution. 10 μL of the reaction mixture is then spotted onto a P30 filtermat and washed three times for 5 minutes in 75 mM phosphoric acid and once in methanol prior to drying and scintillation counting.

### 3.5 PKC Kinases (h)

In a final reaction volume of 25 PKC kinase (h) (5-10 mU) is incubated with 20 mM HEPES pH 7.4, 0.03% Triton X-100, 0.1 mM, 0.1 mg/mL phosphatidylserine, 10 μg/mL diacylglycerol, 0.1 mg/mL histone H1 or 50 μM of the peptide ERMRPRKRQGSVRRRV (SEQ ID No. 20), 10 mM MgAcetate and [γ-₃₃P-ATP] (specific activity approx. 500 cpm/pmol, concentration as required). The reaction is initiated by the addition of the MgATP mix. After incubation for 40 minutes at room temperature, the reaction is stopped by the addition of 5 μL of a 3% phosphoric acid solution. 10 μL of the reaction is then spotted onto a P30 filtermat and washed three times for 5 minutes in 75 mM phosphoric acid and once in methanol prior to drying and scintillation counting.

### 3.6 Results

The results are shown below in Table 1.

TABLE 1
Percentage inhibition of kinase activity
Dendrimer Dend 10-10(4)DP
(20 μM)

| Kinase | % Kinase inhibition* |
|---|---|
| c-RAF | 93 |
| ERK2 | 37 |
| MEK1 | 90 |
| PKB alpha | 29 |
| PKB beta | 85 |
| PKB gamma | 96 |
| PKC alpha | 98 |
| PKC beta I | 98 |
| PKC beta II | 83 |

*Relative to control

As can be seen from Table 1, substantial inhibition of the activity of c-RAF, ERK2, MEK1, PKB beta, PKB gamma, PKC alpha, PKC beta I and PKC beta II was obtained by the peptide dendrimer Dend 10-10(4)DP at a conc. of 20 μM. The peptide dendrimer Dend 10β5 (20 μM) was also found to inhibit the activity of both ERK2 and MEK1 (96% and 92% inhibition, respectively).

### Example 4

### Inhibition of Kinase Activity by Dendrimer Dend 10-10(4)DP

In a dose response study, the inhibition of the activity of the kinases listed Table 2 at increasing concentrations of dendrimer Dend 10-10(4)DP (see Example 2.3) was assessed employing the treatment protocols described in Examples 1 to 3. As shown in the table, inhibition of kinase activity was obtained by the dendrimer at all the concentrations tested. The IC₅₀ percentage inhibition of activity for all the kinases was in the range of from 0.25-2.0 μM. The results shown in Table 2 are percentage inhibition relative to controls. Comparative dose results for 1 μM dendrimer Dend 10-10(4) are also shown.

TABLE 2
Percentage inhibition of kinase activity by dendrimer

| | Percentage (%) kinase inhibition | | | | | | | | |
|---|---|---|---|---|---|---|---|---|---|
| Dendrimer | | PKB | | | PKC | | | PI3K | PI3K |
| Conc. | PKB beta | gamma | PKC alpha | PKC beta I | beta II | MEK1 | c-RAF | P110β | P110δ |
| 250 nm | 53 | 94 | 23 | 19 | 22 | 2 | 64 | 28 | 9 |
| 500 nm | 66 | 98 | 45 | 35 | 44 | 34 | 74 | 34 | 25 |
| 1 μM | 68 | 93 | 83 | 80 | 82 | 76 | 79 | 39 | 39 |
| 2 μM | 70 | 92 | 98 | 96 | 95 | 88 | 81 | 53 | 56 |
| Dend | 70 | 99 | 78 | 67 | 75 | 83 | 71 | 61 | 56 |
| 10-10(4) | | | | | | | | | |
| 1 μM | | | | | | | | | |

In another study using treatment protocols described in Example 1, substantially complete inhibition of the activity of c-Lyn (96%) and c-Yes (99%) relative to controls was obtained by the Dend 10-10(4)DP dendrimer at a concentration of 20 μM. In contrast, as described in Example 1, only relatively low level inhibition of the activity of these kinases compared to c-Src was obtained by the peptide RSKAKNPLYR (SEQ ID No. 6) alone (see also FIG. 5).

While, no increase in the inhibition of c-Src activity was obtained by dendrimer Dend 10-10(4)DP compared to peptide RSKAKNPLYR (SEQ ID No. 6) alone, significant inhibition of MEK1 is obtained by the dendrimer (e.g., see Table 2) whereas negligible inhibition of that kinase was obtained by peptide RSKAKNPLYR (SEQ ID No. 6) at a concentration of 50 μM.

In contrast, dendrimer Dend 10-10(4)DP at a concentration of 20 μM was without any effect on the activities of mTOR, JNK or FAK (data not shown).

### Example 5

### Inhibition of c-Src by Integrin β2, β3, β5 and β6 Based Peptides

Comparison of percentage c-Src inhibition relative to control for the peptides listed in Table 3 was performed utilising the protocol described in Example 1.1. All peptides were tested at a concentration of 50 μM.

TABLE 3
Percentage inhibition of c-Src tyrosine activity

| Peptide | | % Inhibition |
|---|---|---|
| No. | Peptide | of control |
| 1 | RSKAKNPLYR | 57 |
| | (SEQ ID No. 6) | |
| 2 | KEKLKNPLFK | 55 |
| | (SEQ ID No. 9) | |
| 3 | RARAKNPLYK | 29 |
| | (SEQ No. 7) | |
| 4 | RSRARNPLYR | 37 |
| | (SEQ ID No. 8) | |
| 5 | RSKAKWQTGTNPLYR | 44 |
| | (SEQ No. 2) | |
| 6 | RSKAK | 3 |
| | (SEQ ID No. 21) | |
| 7 | WQTGT | 0 |
| | (SEQ ID No. 11) | |
| 8 | NPLYR | 0 |
| | (SEQ ID No. 22) | |

When coupled to the partial signal peptide AAVALLPAVLLALLA (SEQ ID No. 15) the inhibition of c-Src activity for peptides RSKAKNPLYR (SEQ ID No. 6), KEKLKNPLFK (SEQ ID No. 9), RARAKNPLYK (SEQ No. 7) and RSRARNPLYR (SEQ ID No. 8) increased to a range of from 71% to 84%. No inhibitory activity was observed for intervening amino acid linker sequence WQTGT (SEQ ID No. 11) of the β6 binding domain for ERK2 (RSKAKWQTGTNPLYR (SEQ ID No. 2). Similarly, of the 5 mer RSKAK (SEQ ID No. 21) and NPLYR (SEQ ID No. 22) peptides defining the opposite end regions of the β6 binding domain, negligible or no c-Src inhibitory activity was observed.

### Example 6

### Treatment of ADDP Drug Resistant Ovarian Cancer Cells with Cisplatin or Oxaliplatin in Combination with c-Src Tyrosine Kinase Inhibitor Peptide

6.1 Cell Proliferation (MTT) Assay Single cell suspensions of viable trypsinised cells were seeded into 96-well tissue culture plates at a density of 2×10³ cells per well in a volume of 100 μl of a suitable culture media supplemented with heat inactivated foetal calf serum (FCS) (e.g., Dulbecco's Modified Eagles Medium (DMEM), with 10% v/v FCS, 1% L-glutamine, 2% (v/v) Hepes, and antibiotics). A set of triplicate wells was prepared for each concentration of the peptide dendrimer being tested. Additional sample wells containing untreated cells or media alone were set up in each treatment plate and processed in parallel as reference controls. A zero-time plate of untreated cells and media-alone wells was simultaneously prepared and an MTT assay carried out on this plate at the time of addition of the kinase inhibitor (i.e., polypeptide or dendrimer) to treatment plates. All plates were cultured for 24 hours before addition of the test dendrimer.

To prepare the MTT solution 100 mg of MTT (3-(4,5-dimethylthiazolyl-2)-2,5-diphenyltetrazolium bromide) (Cat #. M-128, Sigma, St Louis Mo.), is mixed with 20 ml of PBS at pH 7.4. The resulting solution is filter sterilized (0.2 μM syringe filter) and stored at 4° C. protected from light until use. MTT substrate is cleaved in growing cells to yield a water insoluble salt. After solubilisation of the salt crystals, a coloured product is produced the measurement of which allows quantitation of the proliferative activity of the cultured cells.

Appropriate concentrations of the kinase inhibitor were prepared by dilution of freshly prepared sterile 1 mM stock solutions into cell culture media to give a final well volume of 200 μl containing 10% v/v FCS. The zero-plate was processed by addition of MTT at this time. Cell culture was continued for a further 24 or 48 hours before addition of 20 μl of MTT in PBS (5 mg/ml, 0.2 um filter sterilised). The MTT cell proliferation assay measures cell proliferation rate and, in instances where cell viability is compromised, the assay indicates a comparative reduction in cell viability. After a 3 hour incubation in the presence of MTT (5% CO₂ in air at 37° C.), plates were centrifuged at 450 g for 5 minutes, supernatant was removed by gentle suction and precipitated tetrazolium salt resuspended into 150 μl DMSO:glycine (0.1M glycine, 0.1M NaCl pH 10.5) (6:1 v/v) solution. Plates were gently vortexed to complete solubilisation of crystalline material and absorbance was read at 550 nm using a microplate reader. Sample data were processed to determine the comparative growth of treated samples relative to untreated controls.

### 6.2 Sensitisation of ADDP Ovarian Cancer Cells to Cisplatin

The efficacy of cisplatin and the peptide AAVALLPAVLLALLARSKAKNPLYR (SEQ ID No: 10) (IK2) alone and in combination against the ADDP human ovarian carcinoma cell line was evaluated using the MTT assay described above. The ADDP cell line has induced resistance to cisplatin (Lu Y et al, 1988) and is derived from the A2780 ovarian cancer cell line which is sensitive to cisplatin. The results are shown in FIG. 6. As can be seen, the IK2 peptide in combination with cisplatin resulted in high level inhibition of the growth of the ADDP cancer cells compared to either cisplatin or IK2 peptide alone. The inhibition of the growth of the cells increased with increasing concentration of the IK2 peptide showing that the peptide sensitises the ADDP cells to treatment with cisplatin. The effect of cisplatin on the cisplatin sensitive ovarian cancer cell line A2780 is also shown in this figure.

As expected, the A2780 cell line was highly susceptible to cisplatin with substantially less inhibition of growth of the ADDP cell line by cisplatin being observed. Greater inhibition of growth of ADDP cells by cisplatin in combination with IK2 compared to cisplatin alone was observed at all concentrations of the IK2 peptide utilised.

### 6.3 Sensitisation of ADDP Ovarian Cancer Cells to Oxaliplatin

ADDP ovarian cancer cells were treated with oxaliplatin and the IK2 peptide alone or in combination, and inhibition of growth of the cancer cells again evaluated utilising the MTT assay described above. Similarly to the results obtained utilising cisplatin, the IK2 peptide in combination with oxaliplatin resulted in high level inhibition of the growth of the ADDP cancer cells compared to either oxaliplatin or IK2 peptide alone. The inhibition of the growth of the ADDP cells again also increased with increasing concentration of the IK2 peptide.

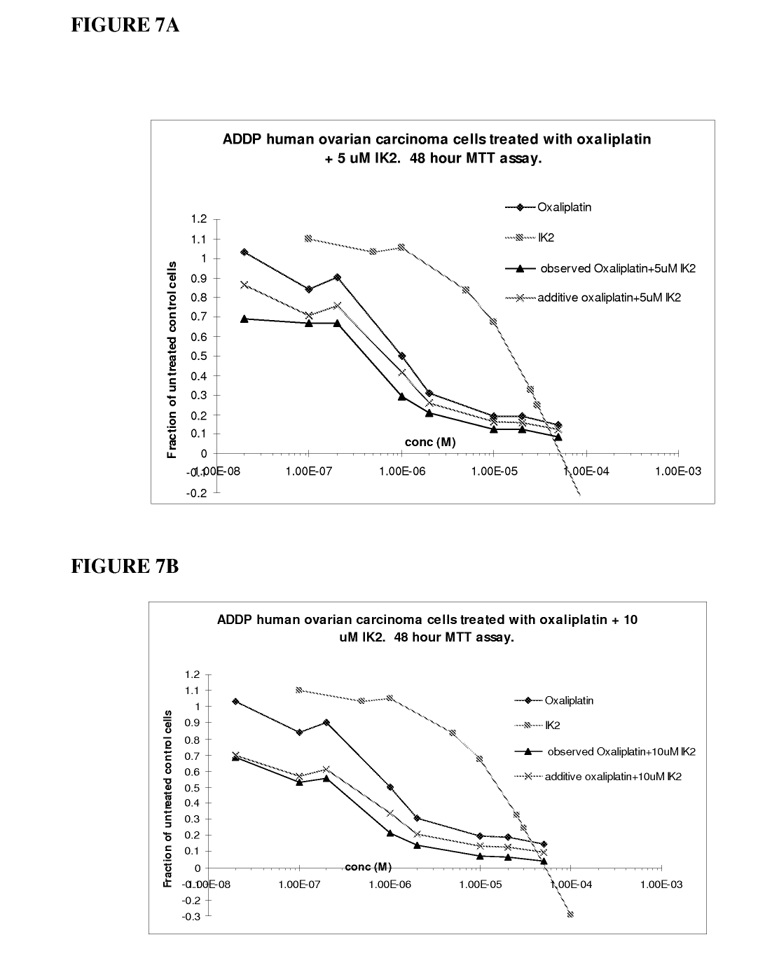

In another study, ADDP ovarian cancer cells were treated with oxaliplatin and 5 μM and 10 μM IK2 peptide either alone or in combination. The results are shown in FIGS. 7A and 7B. The observed inhibition obtained by oxaliplatin in combination with 5 μM or 10 μM IK2 peptide was greater than the calculated additive effect of oxaliplatin and IK2 showing a synergistic outcome was achieved by the oxaliplatin and IK2 peptide combination.

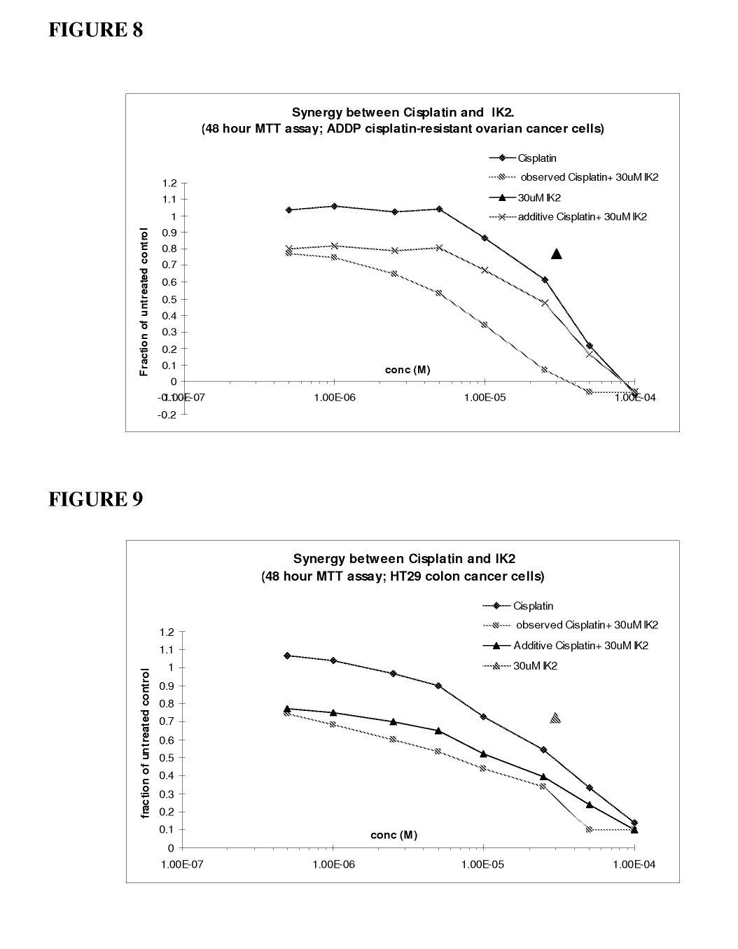

FIG. 8 shows a synergistic effect between cisplatin and 30 μM IK2 peptide against ADDP cisplatin-resistant ovarian cancer cells compared to the calculated additive effect of cisplatin and IK2. A synergistic effect between cisplatin and 30 μM IK2 peptide against HT29 human colon cancer cells compared to the calculated additive effect of cisplatin and IK2 was also found as shown by FIG. 9. A synergistic effect was also obtained with 20 μM RSKAKNPLYR (SEQ ID No. 6) in combination with cisplatin against ADDP cells (72 hour culture) (data not shown).

### Example 7

### Phospho-ERK 1/2 Levels in HT29 Human Colon Adenocaricnoma Cells Treated with Peptide Dendrimers Presenting Peptide RSKAKNPLYR (SEQ ID No. 6)

### 7.1 Cell Culture and Treatment Conditions

Peptide dendrimers of the type illustrated in FIG. 4 comprising lysine branching units presenting either 8 (identified herein as Dend 8-10(4)) or 10 (Dend 10-10(4)) monomer units of the peptide RSKAKNPLYR (SEQ ID No. 6) were utilised.

HT29 cells were harvested using 0.5% trypsin EDTA (Invitrogen) and 5000 cells in 200 μL media were plated into each well of a clear NUNC tissue culture treated 96 well plate (NUNC). Cells were seeded in DMEM media (Invitrogen) supplemented with 10% (v/v) heat inactivated foetal calf serum (FCS, Invitrogen), 1% (v/v) L-glutamine (Invitrogen) and 2% (v/v) 1M Hepes buffer solution (Invitrogen). Cells were then incubated overnight at 37° C.

The following day, the growth medium was replaced with 100 μL of DMEM supplemented with 1% (v/v) L-glutamine and 2% (v/v) 1M HEPES buffer solution (Invitrogen) (serum free medium, SFM) and plates incubated for a further 24 hours at 37° C.

The dendrimers were re-constituted in SFM and the desired concentration was added to the plate in 100 μL SFM to make the total volume of each well 200 μL. The control wells (minus peptide) had only SFM added. The assay plate was then incubated at 37° C. for 4 hours. In studies in which cells were subjected to stimulation by serum, 22 μL FCS (10% v/v final concentration) was added to wells for the final 10 minutes at 37° C. SFM only (22 μL) was added to control wells.

Phospho-ERK levels were evaluated by ELISA utilising an Active Motif FACE ERK1/2 ELISA kit (Australian Biosearch, WA, Australia) as per the manufacturers instructions. Briefly, media was replaced and cells fixed with 4% formaldehyde in PBS, and after a one hour incubation with antibody blocking buffer (supplied), the primary phospho-ERK antibody was added and the plate incubated overnight at 4° C. Antibody dilution buffer only was added to control test wells containing no primary antibody. The following day, HRP-conjugated secondary antibody was added to all wells for one hour before plates were developed and the absorbance measured at 450 nm using a Labsystems Multiskan EX microplate reader (Labsystems, Thermo Labsystems, UK).

### 7.2 ERK1/2 Activation Upon Serum Stimulation is Inhibited by Dendrimeric Peptide

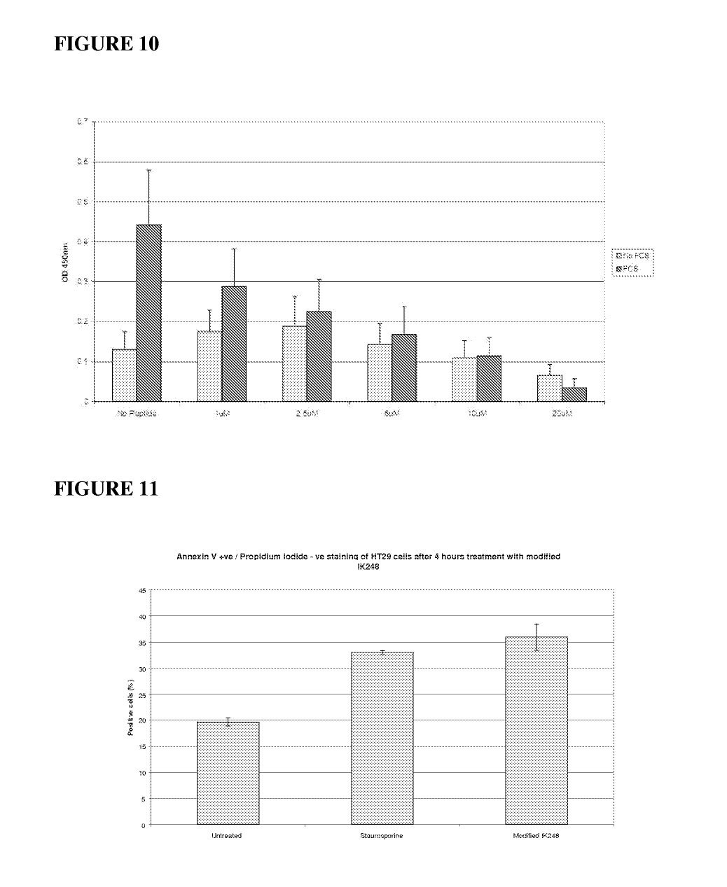

It has previously been reported that MAP kinase activity is dramatically increased in serum-starved HT29 colon cancer cells upon the addition of 10% heat inactivated fetal calf serum (FCS) (Ahmed N et al, Oncogene, 2002; 21: 1370-1380). In the present study, the ability of a dendrimer presenting 8 monomeric units of RSKAKNPLYR (SEQ ID No. 6) (designated Dend 8-10(4)) to inhibit ERK1/2 activation in HT29 cells upon serum stimulation was investigated. As shown in FIG. 10, exposure of the cells to the dendrimer for 4 hours prior to addition of FCS for the last 30 minutes abolished ERK1/2 activation in a dose-dependent manner and similar results were observed for dendrimer Dend 10-10(4) (a dendrimer presenting 10 monomer units of RSKAKNPLYR (SEQ ID No. 6)) (data not shown).

Further, phospho-ERK1/2 levels upon FCS stimulation of HT29 cells were significantly reduced in the presence of Dend 8-10(4) compared to much less inhibition of ERK1/2 activity upon FCS stimulation for skin fibroblast cells, thereby indicating substantial selectivity of the dendrimer for cancer cells compared with normal cells (data not shown). In another study, a dendrimer of the same type but presenting 8 monomeric units of a scrambled form of the RSKAKNPLYR (SEQ ID No. 6) peptide was relatively ineffective at inhibiting ERK1/2 activation upon FCS stimulation in HT29 colon cancer cells compared with the Dend 8-10(4) dendrimer presenting the unscrambled monomeric peptide (data not shown).

### Example 8

### Effect of Peptide Dendrimers on Activity ERK2 and Src Family Kinases in a Cell-Free System

The activity of human c-Src, c-Yes and c-Lyn was assessed as described in Example 1. The activity of c-Fyn was assessed as follows. In a final reaction volume of 25 μL, Fyn (h) (5-10 mU) is incubated with 50 mM Tris pH 7.5, 0.1 mM EGTA, 0.1 mM Na3VO4, 250 μM KVEKIGEGTYGVVYK (SEQ ID No. 19) (Cdc2 peptide), 10 mM MgAcetate and [γ-33P-ATP] (specific activity approx. 500 cpm/pmol, concentration as required). The reaction is initiated by the addition of the MgATP mix. After incubation for 40 minutes at room temperature, the reaction is stopped by the addition of 5 μL of a 3% phosphoric acid solution. 10 μL of the reaction is then spotted onto a P30 filtermat and washed three times for 5 minutes in 75 mM phosphoric acid and once in methanol prior to drying and scintillation counting.

As shown in Table 4, the dendrimer Dend 10-10(4) (presenting 10 monomer units the peptide RSKAKNPLYR (SEQ ID No. 6)) was found to inhibit c-Src activity to a level of 57% and ERK activity to a level of 53%. However, surprisingly c-Src activity was stimulated to 305% of the control (three times the control value) by the dendrimer Dend8-10(4) (presenting 8 monomer units of RSKAKNPLYR (SEQ ID No. 6)). This stimulation of c-Src activity occurred concomitant with significant inhibition of ERK2 activity by the Dend 8-10(4) dendrimer (Table 4).

TABLE 4
Non-cell based assay for ERK2 and c-SRC activity

| Kinase | Control | Dend 8-10(4) | Dend 10-10(4) |
|---|---|---|---|
| ERK2 | 100 | 44 | 53 |
| c-Src | 100 | 305 | 57 |

When a dendrimer in which the RSKAKNPLYR (SEQ ID No. 6) peptides were pegylated with 2 ethylene glycol units and the amino acid residues of the peptide entirely substituted for D-amino acid isomers (designated dendrimer Dend 10-10(4)DP) was used, this dendrimer (at a concentration of 20 μM) was found to inhibit ERK2 activity to a level of 37% and c-Src to a level of 41%. However, at the same concentration of dendrimer Dend 10-10(4)DP, c-Lyn and c-Yes activities were inhibited to levels of 96% and 99%, respectively, of control levels, whereas the activity of c-Fyn was inhibited to a level of 16%.

Further, a dendrimer identical to Dend 10-10(4)DP but presenting the peptide RSRARNPLYR (SEQ ID No. 8) derived from the cytoplasmic binding domain of the β5 integrin subunit for ERK2 (at a concentration of 20 μM) inhibited c-Src activity to a level of 40% and ERK2 activity to a level of 96%. In contrast, only relatively low level inhibition of ERK2 activity was obtained by the peptide RSRARNPLYR (SEQ ID No. 8) monomer alone (4%) relative to control (at a concentration of 50 μM).

### Example 9

### ERK/c-Src Cell Adhesion Assay

The c-Src oncoprotein is extremely potent at causing rapid transformation in cell culture and is over-expressed and activated in many human epithelial malignancies, particularly breast, pancreatic and colon cancers. Activated c-Src induces cellular invasion through a number of effectors, i.e., GTPase Rho and atypical protein kinase C. Moreover, one of the major alterations found in cells transformed by c-Src is that they can proliferate in the absence of external growth factors. One of the consequences of elevated Src activity in colon cancer cells, for example, is disruption of E-cadherin-associated cell-cell contacts. The ability of Src to suppress E-cadherin localisation and function at cell-cell contacts is dependant on Src-induced assembly of integrin adhesion complexes at the tips of membrane protrusions (Avizienyte, E., (2002)).

### 9.1 Methods

Experimental wells were coated with 100 μL of fibronectin (1-10 μg/mL, Sigma-Aldrich) or 100 μL of 1% BSA (negative control, Sigma-Aldrich). Plates were then incubated at 37° C. for 1 hour before being washed with 100 μL PBS. 100 μL of 1% BSA was then added to each well for 30 minutes to block non-specific binding (37°). HT29 human colon cancer cells were harvested using 0.5% trypsin-EDTA and 50,000 cells were plated into each empty well in 100 μL of serum free media (SFM; DMEM supplemented with 1% (v/v) L-glutamine and 2% (v/v) 1M HEPES solution). 100 μL of SFM with or without PP2 (final concentration 5 μM, specific c-Src inhibitor, Sigma-Aldrich), was then added to each well. Plates were centrifuged at 200 rpm for 5 minutes and then incubated at 37° C. for one hour. Phospho-ERK1/2 levels in the cell groups was evaluated by ELISA (Active Motif assay kit, Australian Biosearch) essentially as described in Example 7.1.

### 9.2 Results

Increasing levels of phospho-ERK1/2 in HT29 cells correlated with increasing levels of fibronectin in the absence of serum stimulation. However, the c-Src inhibitor PP2 markedly reduced phospho-ERK levels in all of the treatment groups compared to the control cells (results not shown).

### Example 10

### ERK/PI3K Cell Adhesion Assay

A study was undertaken to evaluate the effect of the PI3K inhibitor Wortmannin (0.25 μM final concentration) on cell adhesion mediated ERK activation in HT29 human colon cancer cells. Phospho-ERK1/2 levels were measured as an indicator of ERK activation substantially as described in Example 7.1. Increasing levels of phospho-ERK1/2 in HT29 cells correlated with increasing levels of fibronectin in the absence of serum stimulation of the HT29 cells. The PI3K inhibitor Wortmannin (0.25 μM) effectively reduced cell adhesion mediated ERK activation by about 55%.

### Example 11

### Induction of Apoptosis in HT-29 Human Adenocarcinoma Cells

The ability of the peptide dendrimer Dend 10-10(4)DP described in Example 2.3 to induce apoptosis in HT-29 human colon cancer cells was assessed. The protocol used in this study is described below and the results are shown in FIG. 11.

### 11.1 Materials

RPMI 1640 cell culture medium, foetal calf serum (FCS), PBS and HBSS (Invitrogen Australia, Mt Waverley, VIC, Australia). Penicillin-streptomycin and Trypan Blue (Sigma-Aldrich, Castle Hill, NSW, Australia). FACScalibur flow cytometer (Becton-Dickinson, North Ryde, NSW, Australia). FITC Annexin V Apoptosis Detection Kit II (BD Pharmingen, North Ryde, NSW, Australia).

11.2 Cell lines

The human colorectal adenocarcinoma cell line HT-29 was sourced from the American Type Culture Collection (ATCC) (Rockville, Md., USA).

### 11.3 Cell Production

HT-29 cells were cultured in RPMI 1640 cell culture medium, supplemented with 10% v/v heat inactivated (FCS) and 50 IU/mL penicillin-streptomycin. All cells were grown at 37° C. in a humidified cell culture incubator supplied with 95% air/5% CO₂. The cells used in this study were used after passage 3.

### 11.4 Cell Seeding

The cells were harvested by trypsinisation, washed twice in HBSS and counted counted using Trypan Blue staining. The cells were then re-suspended in the appropriate culture medium to a concentration of 1×10⁶ cells/mL. A 1 mL volume of this cell dilution was added to 3 wells of a 6 well plate. The cells were allowed to attach to the plate for 1 h.

### 11.5 Compound Formulation

The peptide dendrimer Dend 10-10(4)DP was resuspended in PBS to give a concentration of 150 μM. A 67 μL volume of this dilution was added to 1×10⁶ HT-29 cells in 1 mL of cell culture medium to give a final concentration of 10 μM. Staurosporine was resuspended in DMSO to give a concentration of 1 mM. 10 μL of this dilution was added to 1×10⁶ cells in 1 mL of cell culture medium to give a final concentration of 10 μM.

### 11.6 Apoptosis Assay

1×10⁶ HT-29 cells were incubated in the presence of either Dend 10-10(4)DP or Staurosporine (positive control) for 4 hours at 37° C. in a humidified cell culture incubator supplied with 95% air/5% CO₂. Cells were then harvested by trypsinisation, washed twice in cold phosphate buffered saline (PBS) and then resuspended in 1× binding buffer. 100 μL of the cell suspensions containing 1×10⁵ cells were transferred to a 5 mL plastic tube. A 5 μL volume of FITC annexin V and 5 μL of propidium iodine (PI) were added to each tube and incubated for 15 minutes at room temperature in the dark. 400 μL of 1× binding buffer were added to each tube prior to FACS analysis using a FACScalibur flow cytometer. Three controls were also analysed: unstained cells, cells stained only with Annexin V and cells stained only with PI. The data was presented as the percentage of cells being either Annexin V positive only (excluding PI positive cells), PI positive (total) or Annexin V and PI positive (double stain).

### 11.7 Results

As shown in FIG. 11, a substantial level of apoptosis was observed in HT-29 cells treated with the peptide dendrimer Dend 10-10(4)DP compared to the untreated control group. Similar levels of apoptosis were observed between the staurosporine or peptide dendrimer treatment groups.

### Example 12

### Immunofluorescence Study

HT29 colon cancer cells suspended in RPMI cell culture medium supplemented with 10% FCS, 100 IU/ml penicillin-streptomycin, 2 mM L-Glutamine and 1 mM sodium pyruvate were seeded into 35 mm Fluorodishes and incubated at 37° C. in 95% air/5% CO₂. After 24 hours in culture, 10 μL of either FITC conjugated Dend 10-10(4)DP dendrimer (see Example 2.3) (Dend 10-10(4)DP-FITC (resuspended in phosphate buffered saline) or 10 μL of FITC alone (resuspended in DMSO) were added to separate dishes to give a final compound concentration of 1 μM per dish. The treated dishes were incubated at 37° C. for a further hour and the Hoechst nuclear stain 33258 (Invitrogen) added to the cells 20 minutes prior to imaging at a final concentration of 2.5 μg/ml.

Confocal microscopy was performed using a Nikon C1-Z laser-scanning confocal system equipped with a Nikon E-2000 inverted microscope and three solid laser lines (Sapphire 488 nm, Compass 532 nm and Compass 405 nm). A Nikon 60× water-immersion lens (NA=1.2) objective was used. The samples were imaged with two separate channels (Photomultiplier tubes, PMT). Green fluorescence was excited with an Ar 488 laser line and the emission viewed through BA 495-520 nm narrow band filter in PMT1. The DAPI was excited with a UV 405 nm laser line and the emission viewed through BA 410-465 narrow band filter in PMT2. Signals from PMT1 and PMT2 were merged with the Nikon C1-Z software.

The confocal microscopy showed the FITC-conjugated dendrimer was located at the cell membrane, within the cytoplasm and within nuclei after 1 hour exposure to the compound, in contrast to lack of cellular fluorescence for cells exposed to FITC alone.

### Example 13

### Peptide Dendrimer Comprising 4 or 8 Monomer Units of the Polypeptide RSKAKNPLYR (SEQ ID No. 6) Inhibits the Proliferation of HT29 Colon Cancer Cells

Peptide dendrimers of the type shown in FIG. 4 comprising 4 (Dend 4-10(4)) or 8 (referred to herein as dendrimer Dend 8-10(4), see Example 7.1) monomer units of the polypeptide RSKAKNPLYR (SEQ ID No. 6) were found to inhibit proliferation of HT29 colon cancer cells as assessed by the MTT assay described in Example 6.1. Notably, Dend 8-10(4) was found to be substantially more effective at inhibiting cell growth/proliferation than the peptide dendrimer comprising 4 monomers of the polypeptide (24 hour incubation period).

In another study, the Dend 10-10(4)DP dendrimer also inhibited growth/proliferation of MKN45 gastric carcinoma cells (Cancer Research Laboratory, University of New south Wales, Sydney, Australia), MCF-7 breast cancer cells (breast adenocarcinoma cells obtained from the American Type Culture Collection, ATCC, Manassas Va., United States), and DU145 prostate cancer cells. In this study, the cells were incubated in the presence of the dendrimer for 48 hours. Cell lines were maintained at 37° C. in a humid atmosphere containing 5% CO₂. Cells were passaged at pre-confluent densities using a solution containing 0.05% trypsin and 0.5 mM EDTA (Invitrogen).

### Example 14

### Phospho-ERK1/2 Levels in HT29 Human Colon Cancer Cells Treated with Various Agents

The effectiveness of the peptide dendrimer Dend8-10(4) described above in inhibiting growth factor mediated activation of ERK 1/2 in HT29 cancer cells (i.e., (FCS stimulated) compared to various agents comprising the polypeptide RSKAKNPLYR (SEQ ID No. 6) coupled to different peptide facilitator moieties for facilitating passage of the polypeptide across the plasma cell membrane into the cytosol of the cells was evaluated. Phospho-ERK1/2 levels in the cells following treatment with 5 μM Dend 8-10(4) for 1 hour or polypeptide-facilitator moiety agents (each at 5 μM for the same duration) were measured essentially as described in Example 7.1. The facilitator moieties utilised were the signal peptide fragment AAVALLPAVLLALLA (SEQ ID No. 15), the TAT-G peptide GRKKRRQRRRPPQG (SEQ ID No. 23), a modified pentratin sequence Tr-Pen RRQKWKKG (SEQ ID No. 24), and the penetratin peptide RQIKIWFQNRRMKWKKC_{S-S} (SEQ ID No. 25) wherein S-S indicates a disulphide bridge between the adjacent cysteine residues.

The percentage inhibition of activated phospho-ERK 1/2 by dendrimer Dend 8-10(4) and the polypeptide-facilitator moieties in the HT29 cells at the 1 hour time point is shown in As can be seen, the Dend 8-10(4) dendrimer exhibited at least 30% greater inhibition than the test agent which displayed the closest level of inhibition, namely the TAT-G RSKAKNPLYR (SEQ ID No. 6) polypeptide. At 4 hours, 5 μM Dend 8-10(4) exhibited approx. 95% inhibition of activated phospho-ERK1/2 compared to relatively low level inhibition by the polypeptide-facilitator moieties (data not shown).

### Example 15

### Inhibition of Proliferation in HT29 Human Adenocarcinoma Cells

HT29 cells were cultured for 48 hours in the presence of selected dendrimers and proliferation of the cells was assessed by MTT assay essentially as described in Example 6.1. The results were calculated as percentage proliferation of control cells (not treated with dendrimer).

### 15.1 Inhibition of Proliferation by Dendrimer Dend 10-10(4)

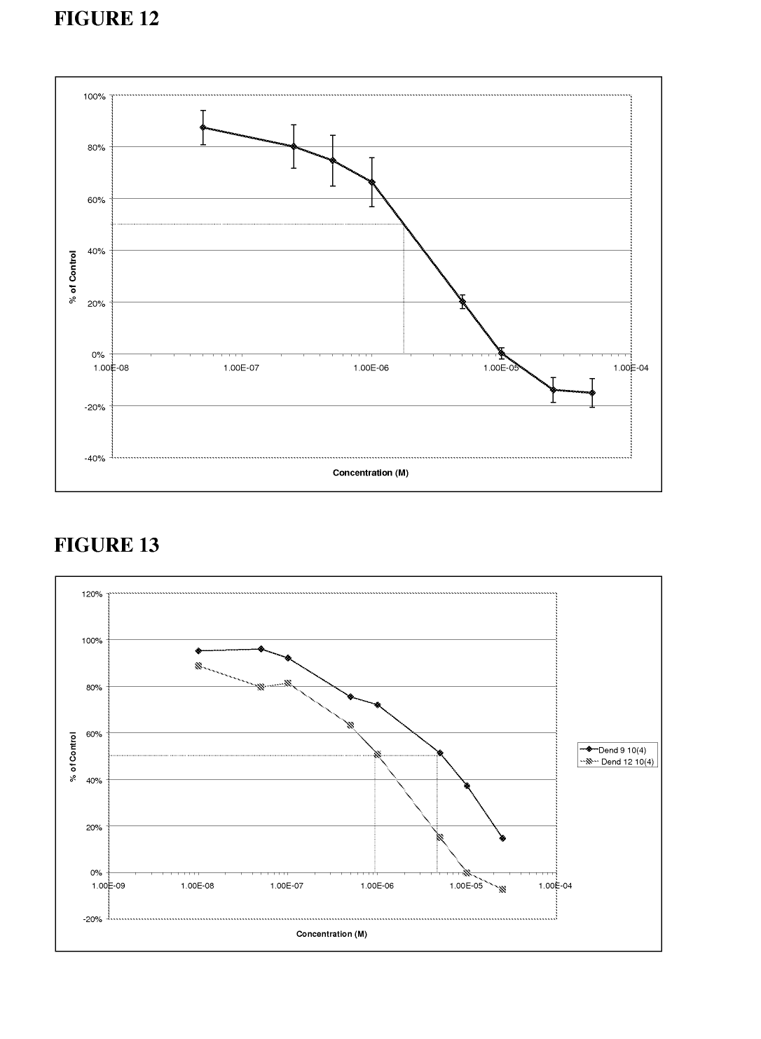

HT29 cells were treated with peptide dendrimer Dend 10-10(4) and the results are shown in FIG. 12. As can be seen, proliferation of the cells was inhibited by the dendrimer.

### 15.2 Dendrimer Size

Dendrimers of the type Shown in FIG. 4 presenting 9 (Dend 9-10(4) or 12 (Dend 12-10(4)) monomer units of the peptide RSKAKNPLYR (SEQ ID No. 6) were assessed for capacity to inhibit proliferation of the HT29 cells. As shown in FIG. 13, Dend 12-10(4) was more effective than Dend 9-10(4) in inhibiting proliferation of the cells. When compared to dendrimer Dend 10-10(4) (presenting 10 monomer units of peptide RSKAKNPLYR (SEQ ID No. 6)) Dend 12-10(4) showed a small improvement in IC₅₀ value (1 μM versus 1.8 μM) but no increase in the dendrimer concentration required for total kill (namely 10 μM for both Dend 12-10(4) and Dend 10-10(4)) was obtained. Dendrimer Dend 10-10(4) was in turn more effective than dendrimer Dend 8-10(4) (presenting 8 monomer units of the RSKAKNPLYR peptide (SEQ ID No. 6)) (IC₅₀'s of 1.8 μM and 5 μM, respectively).

### 15.3 Use of Peptide RSKAKNPLYR (SEQ ID No. 6) Composed of D Amino Acids

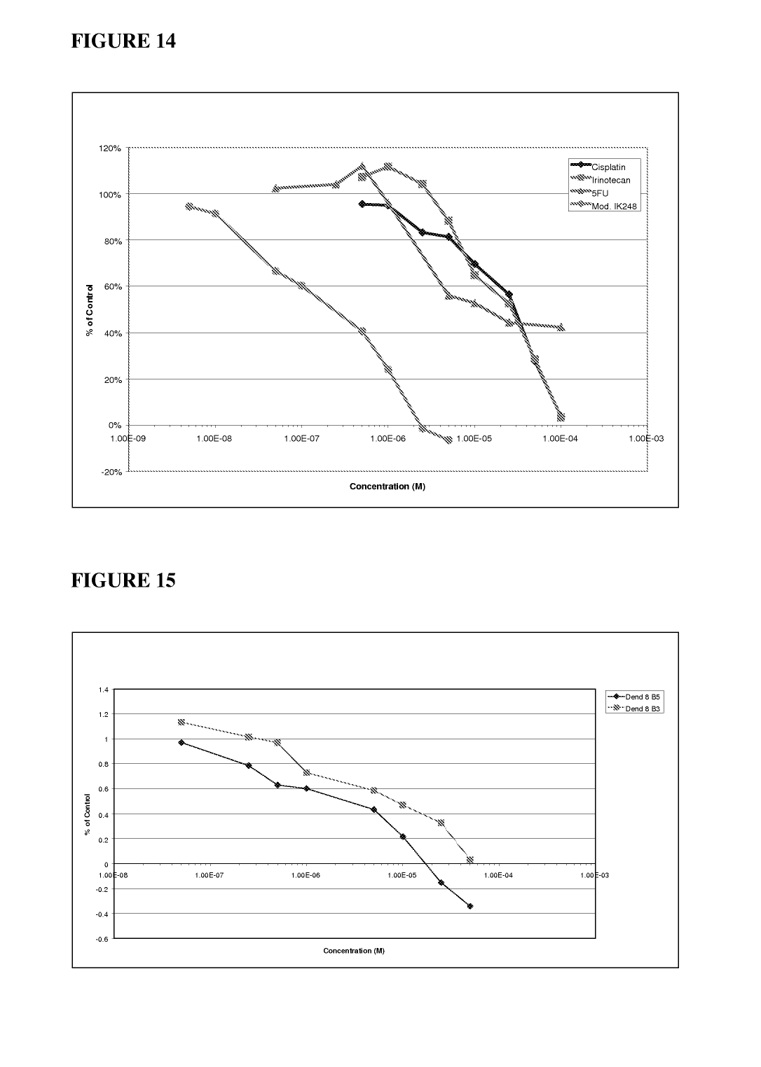

The efficacy of the peptide dendrimer Dend 10-10(4) in which the monomer units of the RSKAKNPLYR peptide (SEQ ID No. 6) were composed entirely of D amino acids and pegylated with two polyethylene glycol (PEG) units at their N-terminal end (identified as Dend 10-10(4)DP) in inhibiting proliferation of human HT29 cells was compared to cisplatin, irinonectin (CPT-11) and 5-fluorouracil (5FU). Proliferation of the cells was assessed by MTT assay and the results are shown in FIG. 14. As can be seen, the Dend 10-10(4)DP dendrimer (identified as Mod. IK248) effected substantially greater inhibition of proliferation of the cells than cisplatin, CPT-11 and 5FU alone.

In another study, exposure of HL60 leukemic cells to the Dend10-10(4)DP dendrimer resulted in inhibition of proliferation of the cells with an IC₅₀ of 2 μM as assayed via MTT assay (data not shown).

### Example 16

### Treatment of HT29 Colon Cancer Cells with Peptide Dendrimers Presenting RARAKNPLYK (SEQ ID No. 7) or RSRARNPLYR (SEQ ID No. 8)

HT29 colon cancer cells were treated with peptide dendrimers of the type illustrated in FIG. 4 presenting 8 monomer units of the 10 mer β3 based peptide RARAKNPLYK (SEQ ID No. 7)) (Dend 8-β3) or the β5 based peptide RSRARNPLYR (SEQ ID No. 8) (identified as Dend 8-β5). Test cells were exposed to the dendrimers for 48 hours and proliferation of the cells was evaluated using the MTT assay essentially as described above in Example 6.1. Absorbance was read at 550 nm using a microtitre plate reader, and the percentage inhibition of proliferation of the test cells was calculated relative to untreated control cells. The results are shown in FIG. 15. As can be seen, both of dendrimers Dend 8-β3 and Dend 8-β5 inhibited proliferation of the HT29 cancer cells, although Dend 8-β5 was more effective.

In another study, a peptide dendrimer of the above type presenting 10 monomer units of the β5 derived peptide RSRARNPLYR (SEQ ID No. 8) consisting entirely of D amino acids inhibited proliferation of HT29 colon cancer cells (MTT assay) with an IC₅₀ of 300 nM. At a concentration of 1 μM, this dendrimer was also shown to inhibit the activity of a range of kinases (relative to control) as shown in Table 6.

TABLE 6
Percentage inhibition of kinase activity
Percentage (%) kinase inhibition

| PKB | PKB | PKC | PKC | PKC | | | PI3K | PI3K |
|---|---|---|---|---|---|---|---|---|
| beta | gamma | alpha | beta I | beta II | MEK1 | c-RAF | p110β | p110δ |
| 87 | 97 | 53 | 79 | 77 | 95 | 85 | 24 | 61 |

### Example 17

### Inhibition of Tumour Growth in a Mouse Model

### 17.1 Materials

Reagents for the culture of HT-29 human colorectal adenocarcinoma cells were obtained from the following suppliers: RPMI 1640 cell culture medium, FBS and HBSS from Invitrogen Australia (Mt Waverley, VIC, Australia); penicillin-streptomycin, phosphate buffered saline (PBS) and trypan blue from Sigma-Aldrich (Castle Hill, NSW, Australia).

### 17.2 Tumour Cell Production

HT29 human colorectal adenocarcinoma cells were cultured in RPMI 1640 cell culture medium supplemented with 10% v/v heat inactivated FCS and 50 IU/mL penicillin-streptomycin. The cells were harvested by trypsinisation, washed twice in HBSS and counted. The cells were then resuspended in HBSS to a final concentration of 2×10⁷ cells/mL.

### 17.3 Test System

Species: Mouse (Mus musculus)

Strain: BALB/c nu/nu

Source: University of Adelaide (Waite Campus, Urrbrae, SA, Australia)

Total number of animals in study: 20 females

Number of study groups: 2 (1 test, 1 control)

Number of mice per group: 10

Body weight range: 20.61-25.27 g at onset of treatment (mean 22.27 g)

Age range: 10-12 weeks at onset of treatment.

### 17.4 Tumour Inoculation

Prior to inoculation the skin on the injection site (dorsal right flank) was swabbed with alcohol. The needle was introduced through the skin into the subcutaneous space just below the animal's right shoulder, and 100 μL of cells (2×10⁶ cells) were discharged. The treatment of mice began nine days after HT29 cell inoculation, the average tumour volume was 68 mm³ (average variability of 6.1%).

### 17.5 Body Weight and Tumour Measurements

Body weight and tumour dimensions (length and diameter) were measured for all animals on the first day of treatment (day 0) and then three times per week, including the termination day of the study (Day 24).

### 17.6 Administration

Mice were randomized, based on body weight, into two groups of ten mice on Day 0 of the study. The peptide dendrimer Dend 10-10(4) presenting 10 monomer units of the peptide RSKAKNPLYR (SEQ ID No. 6) (see Example 2) was used in this study. The vehicle control (phosphate buffered saline (PBS)) and Dend 10-10(4) dendrimer (20 mg/kg) were each administered by intra-tumoural injection once daily for five consecutive days, beginning on Day 0.

The vehicle control and Dend 10-10(4) dendrimer were administered at a dosing volume of 4.762 mL/kg (100 μL) based on a 21 g mouse. Each animal's body weight was measured immediately prior to dosing. The volume of dosing solution administered to each mouse was calculated and adjusted based on individual body weight.

### 17.7 Sample Collection and Calculations

Tumours were excised from all mice post mortem and weighed. Tumour volume was calculated using the equation:

V (mm³)=length×diameter²×π/6

Tumour variability was calculated using the equation:

Variability (%)=(SEM_{Tumour volume}/Tumour volume_{Average})×100

ΔT/ΔC (%) was calculated using the following equation:

ΔT/ΔC %=(Δvolume_{Treatment}/ΔVolume_{Control})×100

where Δ=change in volume from day 0 to the final measurement day or nominated day of interest.

### 17.8 Statistical Calculations

All statistical calculations were performed using SigmaStat 3.0 (SPSS Australia, North Sydney, NSW, Australia). A two-sample t-test was used to determine the significance in body weight change within a treatment group between day 0 and day 4, and between day 0 and the termination day of the study. A One-Way Analysis of Variance (ANOVA) was performed on tumour volumes measured in all mice at the end of the study. Where significant differences were found in the data, the All Pairwise Multiple Comparison Procedures (Holm-Sidak Method) were performed. A two-sample t-test was used to show a significant difference between the tumour weight data for the vehicle control and Dend 10-10(4) treatment groups. A p value of less than 0.05 was considered significant.

### 17.9 Results

TABLE 5
Tumour volume analysis

| | | Days Post-Initial | | | | | | | | | |
|---|---|---|---|---|---|---|---|---|---|---|---|
| Treatment | | Treatment | | | | | | | | | |
| Group | Parameter | 0 | 2 | 4 | 7 | 11 | 14 | 16 | 18 | 21 | 24 |
| Vehicle | Average | 68.1 | 119.2 | 209.3 | 333.2 | 469.7 | 641.6 | 782.2 | 937.0 | 1143.2 | 1372.6 |
| | Stdev | 18.5 | 82.8 | 57.8 | 107.5 | 173.3 | 237.2 | 241.5 | 278.1 | 286.1 | 372.5 |
| | Sem | 5.9 | 9.1 | 18.3 | 34.0 | 54.8 | 75.0 | 76.4 | 87.9 | 90.5 | 117.8 |
| | Delta avg | 0.0 | 51.1 | 141.2 | 265.1 | 401.6 | 573.4 | 714.1 | 868.8 | 1075.1 | 1304.5 |
| | dT/dC [%] | 100.0 | 100.0 | 100.0 | 100.0 | 100.0 | 100.0 | 100.0 | 100.0 | 100.0 | 100.0 |
| Dend 10- | Average | 68.0 | 136.6 | 211.9 | 248.6 | 300.9 | 323.9 | 391.0 | 476.9 | 601.5 | 702.1 |
| 10(4) | Stdev | 19.8 | 62.6 | 123.7 | 132.1 | 110.8 | 177.5 | 207.2 | 245.7 | 280.9 | 351.9 |
| | Sem | 6.3 | 19.8 | 39.1 | 41.8 | 35.0 | 56.1 | 65.5 | 77.7 | 88.8 | 111.3 |
| | Delta avg | 0.0 | 68.6 | 144.0 | 180.6 | 232.9 | 255.9 | 323.1 | 408.9 | 598.7 | 710.4 |
| | dT/dC [%] | 100.0 | 134.3 | 102.0 | 68.1 | 58.0 | 44.6 | 45.2 | 47.1 | 55.7 | 54.5 |

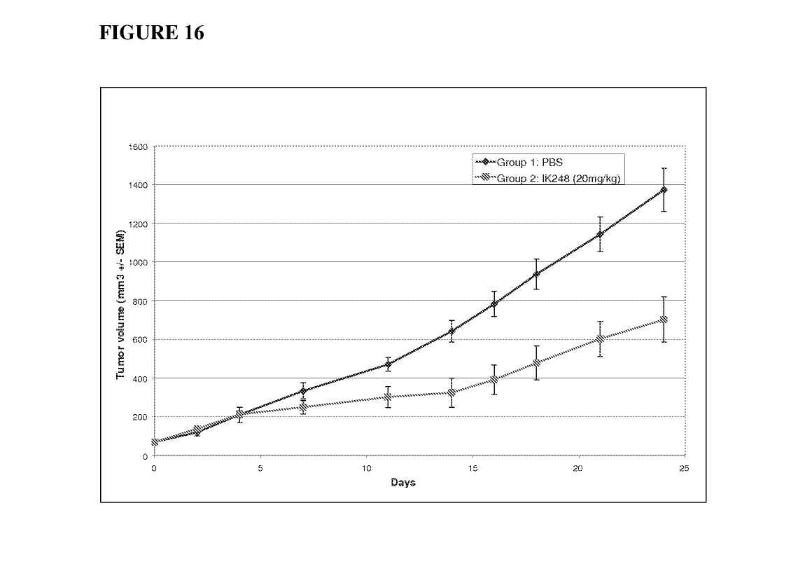

As shown more clearly in FIG. 16, tumour growth was markedly inhibited by the dendrimer Dend 10-10(4) (solid squares, and identified as IK248 in FIG. 16) compared to the vehicle only control (solid diamonds). In particular, the growth of the tumours slowed noticeably between Day 5 to Day 15 in the treatment groups relative to the control group. The average tumour weight in the control group at the end of the study was 0.962 g±0.124 SEM compared to 0.437 g±0.072 SEM for the Dend 10-10(4) treatment group, a highly significant outcome (P≦0.003).

Although a number of preferred embodiments have been described, it will be appreciated by persons skilled in the art that numerous further embodiments may be provided without departing from the invention. The present embodiments described are, therefore, to be considered in all respects as illustrative and not restrictive.

### LITERATURE REFERENCES

1. Abram C L, Courtneidge S A (2000), Exp Cell Res 254: 1-13.
2. Agrez, M. V. and Bates R. C., Colorectal cancer and the integrin family of cell adhesion receptors: current status and future directions. European J. Cancer 30A, 2166-2170 (1994).
3. Agrez, M. V., Bates R. C., Mitchell, D., Wilson, N., Ferguson, N., Anseline, P. and Sheppard. D., Multiplicity of fibronectin-binding αv integrin receptors in colorectal cancer. Br. J. Cancer 73, 887-892 (1996).
4. Agrez, M. V., Gu, X., Turton, J., Meldrum, C., Niu, J., Antalis, T. and Howard, E. W., The αvβ6 integrin induces gelatinase B secretion in colon cancer cells. Int. J. Cancer 81, 90-97 (1999).
5. Allgayer H, Boyd D D, Heiss M M, Abdalla E K, Curley S A and Gallick G E (2002), Cancer, 94: 344-351.
6. Avizienyte, E. et al, Src-induced de-regulation of E-cadherin in colon cancer cells requires integrin signalling. (2002) Nature Cell Biology, (On-line reference). DOI: 10.1038/ncb829.
7. Benistant et al, Oncogene; 19: 5083-5090, 2000.
8. Boulton, T. G., Nye, S. H. and Robbins, D. J., ERKs: a family of protein-serine/threonine kinases that are activated and tyrosine phosphorylated in response to insulin and NGF. Cell 65, 663-675 (1991).
9. Breuss, J. M., Gallo, J., De Lisser, H. M., Klimanskaya, I. V., Folkesson, H. G., Pittet, J. F., Nishimura, S. L., Aldape, K., Landers, D. V., Carrenter, W., Gillet, N., Sheppard, D., Mathay, M., Albeda, S. M., Kramer, R. H., and Pytela, R., Expression of the β6 integrin in development, neoplasia and tissue repair suggests a role in epithelial remodelling. J. Cell Sci. 108, 2241-2251 (1995).
10. Browaeys-Poly E, Cailliau K, Vilain J P (2000), Eur J Biochem 267: 6256-6263.
11. Bolen J B, Veillett A, Schwartz A M, DeSeau V and Rosen N (1987), Proc Natl Acad Sci 84: 2251-2255.
12. Budde R J, Ke S, Levin V A (1994), Cancer Biochem Biophys 14: 171-175.
13. Cheng J Q et al, PNAS USA, 1996: 93; 3636-3641.
14. Cheng J Q et al, Oncogene, 2005: 24; 7482-7492.
15. Choi H S, Kim H H, Yang J M and Shin S., An insight into the gene delivery mechanism of the arginine peptide system: Role of the peptide/DNA complex size. Biochimica et Biophysica Acta (BBA), 2006; 1760: 1604-1612.
16. Cloninger, M. J. Biological applications of dendrimers. Curr. Opin. Chem. Biology. 6, 742-748 (2002)
17. Cone, R. I., Weinacker, A., Chen, A. and Sheppard, D., Effects of beta subunit cytoplasmic domain deletions on the recruitment of the integrin alpha v beta 6 to focal contacts. Cell Adhes. Comm. 2, 101-113 (1994).
18. Datta et al, Genes & Development, 13:2905-2927, 1999.
19. De Boer P A, Crossley, R E, and Rothfield, L I. A division inhibitor and topological specific factor coded for by the miicell locus determine proper placement of the division septum in E. coli. Cell, 56; 641-649, 1989.
20. Dennis P A, Targeting Akt in Cancer: Promise, Progress, and Potential Pitfalls. AACR Education Book, 2008: 25-35
21. Erpel T, Alonso G, Roche S, Courtneidge S A (1996), J Biol Chem 271: 16807-16812.
22. Fields A P et al, Cancer Res, 2009: 69(4): 1643-1650.
23. Giancotti, F. G. and Ruoslahti, E., Integrin signalling. Science 285, 1028-1032 (1999).
24. Haier J, Gallick G E, Nicolson G L (2002), J Exp Ther Oncol 2: 237-245.
25. Huveneers S, van den Bout I, Sonneveld P, Sancho A, Sonnenberg A and Danen E H J., (2007), Cancer Res; 67: 2693-2700.
26. Hynes, R. O., Integrins: versatility, modulation and signaling in cell adhesion. Cell 69, 11-25 (1992).
27. Irby R B, Mao W, Coppola D, Kang J, Loubeau J M, Trudeau W, Karl R, Fujita D J, Jove R and Yeatman T J (1999), Nature Genetics 21: 187-190.
28. Ishizawar, R and Parsons, S. J, (2004), C-src and cooperating partners in human cancer, Cancer Cell: 6: 209-214.
29. Kim H H, Lee W S, Yang J M and Shin S., Basic peptide system for efficient delivery of foreign genes. Biochimica et Biophysica Acta (BBA), 2003; 1640: 129-136.
30. Kruger J S and Reddy K B, Mol Cancer res, 2003: 1(11); 801-809.
31. Lee, C. C., MacKay, J. A., Frechet, J. M. and Szoka, F. C. Designing dendrimers for biological applications. Nature Biotech. 23, 1517-1526 (2005).
32. Lu Y, Han J and Scanlon K J (1988), J Biol Chem 263: 4891-4894.
33. Lu Y, Yu Q, Liu J H, et al (2003), J Biol Chem 278: 400057-40066.
34. Lutz M P, Silke Eber I B, Flossman-Kast B B M et al (1998), Biochem Biophys Res Commun 243: 503-508.
35. MacDiamid, JA., Mugridge, NB., and Wiess, J C (2007), Cancer Cell; 11; 431-445.
36. Majumber P K and Sellers W R, Oncogene, 2005: 24; 7465-7474.
37. Miranti C K & Brugge J S (2002), Nat. Cell Biol. 4: E83-E90.
38. Murray N R et al, J Cell BIol, 1999: 145(4); 699-711.
39. Myoui A, Nishimura R, Williams P J et al (2003), Cancer Res 63: 5028-5033.
40. Nanjundan M, Sun J, Zhao J, et al (2003), J Biol Chem 278: 37413-37418.
41. Nakatani K et al, J Biol Chem, 1999: 274; 21528-21532.
42. Needlemen, S. B., and Wunsch, C. C. A general method applicable to the search for similarities in the amino acid sequence of two proteins. J. Mol. Biol. 48(3), 443-53, 1970.
43. Pengetnze Y, Steed M, Roby K F, Terranova P F, Taylor C C (2003), Biochem Biophys Res Commun 309: 377-383.
44. Reszka, A. A., Hayashi, Y. and Horwitz, A. F., Identification of amino acid sequences in the integrin beta 1 cytoplasmic domain implicated in cytoskeletal association. J. Cell Biol. 117, 1321-1330 (1992).
45. Sadler, K. and Tam, J. P. Peptide dendrimers: applications and synthesis. Rev. Mol. Biotechnology. 90, 195-229, (2002).
46. Sato M, Tanaka T, Maeno T et al (2002), Am J Respir Cell Mol Biol 26: 127-34.
47. Sebolt-Leopold J S, Clinical Cancer Res, 2008: 14; 3651-3662.
48. Sheppard, D., Rozzo, C., Starr, L., Quaranta, V., Erle, D. J. and Pytela, R., Complete amino acid sequence of a novel integrin β subunit (β6) identified in epithelial cells using the polymerase chain reaction. J. Biol. Chem. 265, 11502-11507 (1990).
49. Summy & Gallick, (2006), Clin Cancer Res: 2006: 1398-1401.
50. Sun M, Cancer Res, 2001:61(16); 5985-91.
51. Talamonti M S, Roh M S, Curley S A and Gallick G E (1993), J Clin Invest 91: 52-60.
52. Termulen P M, Curley S A, Talamonti M S, Saboorian M H and Gallick G E (1993), J Surg Res 54: 293-298.
53. Venkateshwar G et al, J Biol Chem, 2002:277:22558-22565.
54. Wang Q, J Biol Chem, 200: 278(51); 51091-51099.
55. Weiqun L et al, Oncogene, 1999, 18(47), 6564-6572.
56. Wiener J R, Windham T C, Estrella V C et al (2003), Gynecol Oncol 88: 73-79.
57. Windham T C, Parikh N U, Siwak D R et al (2002), Oncogene 21:7797-7807.
58. Workman et al., Current Opinion Investig. Drugs, 2007:8; 445-446.
59. Zhang et al, Oncogene, 1999: 18(47); 6564-6572.
60. Zhang J et al, J Biol Chem, 2004: 279(21); 22118-22123.
61. Zhao & Roberts, PI3 Kinases in Cancer: From Oncogene Artifact to Leading Cancer Target; Sciences STKE 2006 (365), pe52 (ISSN 1525-8882).

1. A method for inhibiting growth and/or proliferation of a cancer cell, comprising:
selecting at least one inhibitor for inhibiting at least one protein kinase in at least one cell activation pathway of the cancer cell other than a MAP kinase, the inhibitor being a polypeptide providing a cytoplasmic binding domain of a β integrin subunit for binding of ERK2, or a variant or modified form of the binding domain, to which ERK2 binds; and
treating the cancer cell with an effective amount of the polypeptide to inhibit the protein kinase.

2. A method according to claim 1 for inhibiting a plurality of protein kinases other than MAP kinases, to inhibit a plurality of said cell activation pathways in the cancer cell.

3. A method according to claim 1 comprising treating the cancer cell with the polypeptide to inhibit at least one protein kinase selected from the group consisting of c-Raf, MEK1 and kinases in the Src, PI3K, Protein kinase B (PKB/AKT), and Protein kinase C (PKC) families.

4. A method according to claim 1 comprising treating the cancer cell with the polypeptide to inhibit at least one kinase in a cell activation pathway in the cancer cell other than, or besides, the Ras/Raf/MEK/MAPK pathway.

5. A method according to claim 1 comprising treating the cancer cell with the polypeptide to inhibit at least one cell activation pathway selected from the group consisting of the PI3 kinase/AKT pathway, and cell activation pathways including one or more kinases in the Src and/or PKC kinase families.

6. A method according to claim 5 comprising treating the cancer cell with the polypeptide to inhibit at least one cell activation pathway including one or more kinases selected from the group consisting of kinases in the Src, PKB and PKC families.

7. A method according to claim 5 comprising treating the cancer cell with the polypeptide to inhibit a kinase selected from the group consisting of c-Src, c-Yes, c-Lyn, c-Fyn, PKB beta and PKB gamma, PKC alpha, PKC beta I, PKC beta II, and PKC gamma.

8. A method according to claim 5 comprising treating the cancer cell with the polypeptide to inhibit at least one PI3 kinase including a catalytic subunit selected from the group consisting of p110 beta and p110 delta.

9. A method according to claim 1 wherein the binding domain of the β integrin subunit incorporates an intervening amino acid linker sequence that links opposite end regions of the binding domain together and which is not essential for the binding of the MAP kinase, the opposite end regions of the binding domain being defined by respective amino acid sequences.

10. A method according to claim 9 wherein the polypeptide comprises a variant or modified form of the binding domain of the β integrin subunit, and one or more amino acids of the amino acid linker sequence are deleted and/or differ in the polypeptide compared to the binding domain.

11. A method according to claim 10 wherein all of the amino acids in the intervening amino acid sequence are deleted in the polypeptide compared to the binding domain.

12. A method according to claim 9 wherein the amino acid sequence identity of the opposite end regions of the binding domain are unchanged in the polypeptide compared to the binding domain.

13. A method according to claim 1 wherein the polypeptide is coupled to a facilitator moiety for facilitating passage of the polypeptide into the cancer cell.

14. A method according to claim 1 wherein the polypeptide is presented by a dendrimer and the cancer call is treated with the dendrimer.

15. A method according to claim 14 wherein the dendrimer presents more than 8 monomer units of the polypeptide.

16. A method according to claim 15 wherein the dendrimer presents 10 monomer units of the polypeptide.

17. A method according to claim 14 wherein the polypeptide is N- and/or C-terminal protected against protease degradation.

18. A method according to claim 17 wherein the polypeptide is pegylated with a plurality of ethylene glycol units to protect against said protease degradation.

19. A method according to claim 1 wherein the binding domain, or the variant or modified form of the binding domain, presented by the polypeptide includes one or more D-amino acids.

20. A method according to claim 1 being a method for prophylaxis or treatment of cancer in a mammal, and comprising treating the mammal with an effective amount of the polypeptide to the mammal.

21. A method for inhibiting activity of at least one protein kinase, comprising contacting the protein kinase with a polypeptide providing a MAP kinase cytoplasmic binding domain of a β integrin subunit for binding of ERK2, or a variant or modified form of the binding domain, to which ERK2 binds, the protein kinase being selected from the group consisting of c-RAF, MEK1, and kinases in the Src, PI3K, PKB and PKC families.

22. A method for inhibiting a plurality of cell activation pathways in a cancer cell, comprising treating the cancer cell with an effective amount of at least one polypeptide providing a MAP kinase cytoplasmic binding domain of a β integrin subunit for binding of ERK2, or a variant or modified form of the binding domain, to which ERK2 binds.

23. A method for prophylaxis or treatment of cancer in a mammal, comprising administering to the mammal an effective amount of at least one dendrimer for inhibiting a plurality of cell activation pathways in cancer cells of the cancer, the dendrimer presenting at least one polypeptide providing a MAP kinase cytoplasmic binding domain of a β integrin subunit for binding of ERK2, or a variant or modified form of the binding domain, to which ERK2 binds.

24.-25. (canceled)


SEQUENCE LISTING (26 sequences)

SEQ ID NO: 1 - protein, 3 aa, Artificial Sequence
  synthetic construct
RGD

SEQ ID NO: 2 - protein, 15 aa, Artificial Sequence
  synthetic construct
RSKAKWQTGTNPLYR

SEQ ID NO: 3 - protein, 15 aa, Artificial Sequence
  synthetic construct
RARAKWDTANNPLYK

SEQ ID NO: 4 - protein, 15 aa, Artificial Sequence
  synthetic construct
RSRARYEMASNPLYR

SEQ ID NO: 5 - protein, 16 aa, Artificial Sequence
  synthetic construct
KEKLKSQWNNDNPLFK

SEQ ID NO: 6 - protein, 10 aa, Artificial Sequence
  synthetic construct
RSKAKNPLYR

SEQ ID NO: 7 - protein, 10 aa, Artificial Sequence
  synthetic construct
RARAKNPLYK

SEQ ID NO: 8 - protein, 10 aa, Artificial Sequence
  synthetic construct
RSRARNPLYR

SEQ ID NO: 9 - protein, 10 aa, Artificial Sequence
  synthetic construct
KEKLKNPLFK

SEQ ID NO: 10 - protein, 25 aa, Artificial Sequence
  synthetic construct
AAVALLPAVLLALLARSKAKNPLYR

SEQ ID NO: 11 - protein, 5 aa, Artificial Sequence
  synthetic construct
WQTGT

SEQ ID NO: 12 - protein, 6 aa, Artificial Sequence
  synthetic construct
SQWNND

SEQ ID NO: 13 - protein, 5 aa, Artificial Sequence
  synthetic construct
WDTAN

SEQ ID NO: 14 - protein, 5 aa, Artificial Sequence
  synthetic construct
YEMAS

SEQ ID NO: 15 - protein, 15 aa, Artificial Sequence
  synthetic construct
AAVALLPAVLLALLA

SEQ ID NO: 16 - protein, 16 aa, Artificial Sequence
  synthetic construct
AAVALLPAVLLALLAP

SEQ ID NO: 17 - protein, 5 aa, Artificial Sequence
  synthetic construct
DLXXL

SEQ ID NO: 18 - protein, 12 aa, Artificial Sequence
  synthetic construct
RTDLDSLRTYTL

SEQ ID NO: 19 - protein, 15 aa, Artificial Sequence
  synthetic construct
KVEKIGEGTYGVVYK

SEQ ID NO: 20 - protein, 16 aa, Artificial Sequence
  synthetic construct
ERMRPRKRQGSVRRRV

SEQ ID NO: 21 - protein, 5 aa, Artificial Sequence
  synthetic construct
RSKAK

SEQ ID NO: 22 - protein, 5 aa, Artificial Sequence
  synthetic construct
NPLYR

SEQ ID NO: 23 - protein, 14 aa, Artificial Sequence
  synthetic TAT-G peptide
GRKKRRQRRRPPQG

SEQ ID NO: 24 - protein, 8 aa, Artificial Sequence
  synthetic modified pentratin sequence Tr-Pen
RRQKWKKG

SEQ ID NO: 25 - protein, 18 aa, Artificial Sequence
  synthetic penetratin
RQIKIWFQNRRMKWKKCC

SEQ ID NO: 26 - protein, 13 aa, Artificial Sequence
  synthetic construct
GRPRTSSFAEGKK